(19) United States
(12) Patent Application Publication
(10) Pub. No.: US 2021/0239683 A1
(43) Pub. Date: Aug. 5, 2021

(54) CELL ENCAPSULATION COMPOSITIONS AND METHODS FOR IMMUNOCYTOCHEMISTRY

(71) Applicant: The University of British Columbia, Vancouver (CA)

(72) Inventors: Hongshen Ma, Vancouver (CA); Jeong-Hyun Lee, Vancouver (CA)

(73) Assignee: The University of British Columbia, Vancouver, BC (CA)

(21) Appl. No.: 17/050,989

(22) PCT Filed: May 3, 2019
(86) PCT No.: PCT/CA2019/050593

§ 371 Date: Oct. 27, 2020

Related U.S. Application Data
(60) Provisional application No. 62/666,371, filed on May 3, 2018.

(51) Int. Cl. G01N 33/531; G01N 1/30; C08L 71/02; C08L 51/08; C08J 3/075

(52) U.S. Cl. CPC G01N 33/531; G01N 1/30; C08L 71/02; C08L 51/08; C08J 2451/08; C08J 2371/02; C08J 2351/08; C08J 2471/02; C08J 3/075

(57) ABSTRACT

Provided herein are compositions comprising: a scaffold polymer having one or more acryloyl groups or one or more methacryloyl groups; optionally a porogen and a crosslinking agent, compositions that upon crosslinking form a hydrogel for use in cell encapsulation and methods for immunocytochemistry of encapsulated cells. Scaffold polymers used are selected from: Poly(ethylene glycol) diacrylate (PEGDA); Poly(ethylene glycol) dimethylacrylate (PEGDMA); Poly(ethylene glycol) methyl ether acrylate (PEGMEA); Poly(ethylene glycol) methacrylate (PEGMA); and Poly(ethylene glycol) methyl ether methacrylate (PEGMEMA), and porogens selected from: Poly(ethylene glycol) (PEG); Chitosan; Agarose; Dextran; Hyaluronic acid; Poly(methyl methacrylate) (PMMA); Cellulose and derivatives thereof; Gelatin and derivatives thereof; and Acrylamide and derivatives thereof. The invention also provides, at least in part, compositions for forming a porous hydrogel around a cell suitable for immunostaining of cells within the hydrogel.

### CROSS REFERENCE TO RELATED APPLICATION

This application claims the benefit of U.S. Provisional Patent Application Ser. No. 62/666,371 filed on 3 May 2018, entitled “REAGENT AND PROCESS FOR LOSSLESS IMMUNOCYTOCHEMISTRY”.

### FIELD OF THE INVENTION

The invention relates to cell encapsulation compositions and methods for immunocytochemistry. The invention also provides compositions for forming a porous hydrogel around a cell suitable for immunostaining of cells within the hydrogel.

### BACKGROUND OF THE INVENTION

Immunocytochemistry (ICC), or immunofluorescence, are a variety of assays for phenotyping cells based on protein expression and localization established by labeling using antibodies having a detectable tag. An ICC assay will often involve the steps of fixation, permeabilization, blocking, and immunostaining. Each of these steps is followed by at least one washing step, where reagent solutions are exchanged. When working with non-adherent cells, the additional step of centrifuging the cells into a pellet to remove the supernatant by pipetting or pouring^{1,2} is also required and can be time consuming. When there are a large number of cells (>10⁵), a pellet forms easily and has sufficient mass to remain in place during supernatant removal. When there are fewer cells, pelleting becomes more challenging and the smaller mass of cells is more easily lost during supernatant removal. This issue is particularly important when working with precious samples, where the specimen is limited; or when searching for rare cells within a larger number of cells, such as circulating tumor cells (CTCs)³⁻⁶ and fetal cells in maternal blood⁷, where cell loss has more significant consequences.

The need to hold cells in place during washing and supernatant removal is particularly important in automated high-throughput screening systems, where reducing the number of cells in each aliquot dramatically reduces the total sample and increases the throughput of a screening process. In these systems, centrifugation steps often represent a significant bottleneck for processing times. Therefore, an effective method to hold the cells in place during wash steps would reduce the total number of centrifugation steps and dramatically reduce the overall time required for screening.

Numerous adaptations of the conventional ICC protocol have been developed to prevent cell loss. One approach is to attach cells on a glass slide coated using an adhesive, such as poly-L-lysine, fibronectin, or Cell-tak⁸⁻¹⁰, and then perform the ICC protocol on the glass slide. This approach works well for adherent cells grown in culture, but the adhesives are typically ineffective for primary cells or suspension cells grown in culture. Alternatively, another approach is Cytospin™, which physically adheres cells to a glass slide using high centrifugal force^{11,12}. While both primary cells and cultured cells can be effectively adhered to a glass slide, but this process may still result in significant losses. Specifically, when the cell number is relatively small (<10⁵ input cells), previous studies have reported losses of >75%¹³. Furthermore, Cytospin™ is a serial process performed one sample at a time, which has limited capacity for high-throughput screening studies involving large numbers of samples¹⁴. Finally, while Cytospin™ deposits cells in a confined region on a slide, the deposition area is typically very large for microscopy. Consequently, analyzing these cells requires imaging over many microscopy fields in order to detect a sufficient number of cells, which is particularly challenging when searching for rare cells, such as CTCs.

### SUMMARY OF THE INVENTION

This invention is based in part on the surprising discovery that water soluble scaffold polymers having one or more acryloyl group (for example, PEGDA) or one or more methacryloyl groups (for example, PEGDMA), an average molecular weight (Mₙ) of less than or equal to about 6,000, at specific percentages are able to form hydrogels via cross-linking that are able to physically restrain cells in a sample with sufficient mechanical strength to withstand repeated washings, while remaining permeable to immunostaining reagents and have sufficient transparency for a variety of microscopic techniques.

This invention is based in part on the discovery that PEGDA hydrogels can be cross-linked to physically restrain cells in a sample, while remaining permeable to immunostaining reagents. The hydrogels described herein are sufficiently robust to withstand repeated washings, and are compatible with producing high-quality microscopy images.

In another embodiment, there is provided a method of preparing a hydrogel using a hydrogel-forming composition as described herein. The method generally comprises the steps of: 1) Mixing a cell suspension with a hydrogel-forming composition described herein, to create a pre-hydrogel polymer solution; 2) initiating cross-linking by chemical activation or photo-activation. Crosslinking may be photo-activated by exposing a pre-hydrogel polymer solution containing (or in contact with) a photo-initiator to UV and/or visible light Crosslinking may be chemically activated by contacting a pre-hydrogel polymer solution with a chemical initiator and waiting an appropriate amount of time for cross-linking to occur.

In another embodiment, there is provided a method of preparing a hydrogel for immunocytochemistry using a hydrogel-forming composition as described herein. The method generally comprising the steps of: 1) Mixing a cell suspension with a hydrogel-forming composition described herein, to create a pre-hydrogel polymer solution; 2) applying the pre-hydrogel polymer solution to a surface of an imaging container for immunocytochemistry, such as a microtiter plate; 3) centrifuging the imaging container or allowing the cells to settle by gravity to align cells to an imaging surface, 4) cross-linking the pre-hydrogel polymer solution by chemical-activation or photo-activation to form a hydrogel.

In another embodiment, there is provided a method of preparing a hydrogel for immunocytochemistry using a hydrogel-forming composition as described herein. The method generally comprising the steps of: 1) Mixing a hydrogel-forming composition described herein, to create a pre-hydrogel polymer solution; 2) add the pre-hydrogel polymer solution to an imaging container for immunocytochemistry, such as a microtiter plate; 3) add a cell suspension into the imaging container; 4) centrifuging the imaging container or allowing the cells to settle by gravity to align cells to an imaging surface, 5) cross-linking the pre-hydrogel polymer solution by chemical-activation or photo-activation to form a hydrogel.

In another embodiment, there is provided a method of preparing a hydrogel for immunocytochemistry using a hydrogel-forming composition as described herein. The method generally comprising the steps of: 1) Mixing a hydrogel-forming composition described herein, to create a pre-hydrogel polymer solution; 2) add a cell suspension to an imaging container for immunocytochemistry, such as a microtiter plate; 3) add the pre-hydrogel polymer solution to the imaging container; 4) centrifuging the imaging container or allowing the cells to settle by gravity to align cells to an imaging surface, 5) cross-linking the pre-hydrogel polymer solution by chemical-activation or photo-activation to form a hydrogel.

Alternatively, a hydrogel for immunocytochemistry as described herein may be prepared using a pre-deposited crosslinking agent. The method comprising the steps of: 1) pre-depositing (or coating) a surface of an imaging container (for example, plate, or slide etc.) with a crosslinking agent; 2) mixing a cell suspension with a hydrogel-forming pre-hydrogel polymer solution, wherein the pre-hydrogel polymer solution comprises a scaffold polymer, and optionally, a porogen; 3) centrifuging the imaging container to or allowing the cells to settle by gravity to align cells to an imaging surface and to allow contact between the pre-hydrogel polymer solution and the pre-deposited crosslinking agent to initiate crosslinking.

Provided herein is a method of carrying out an immunocytochemistry procedure using the hydrogel-forming compositions described herein. It has been demonstrated that cells can be added to hydrogel-forming compositions of the present invention and encapsulated therein upon hydrogel polymerization. Cells and other biological materials of particular use with the methods of this invention include but are not limited to primary cells, cultured cells, cancer cells, patient-derived cells, circulating tumor cells, stem cells, epithelial cells, endothelial cells, smooth muscle cells, hematological cells, immune cells, reticulocytes, fetal calls, parasites, helminths, bacteria, archaea, spermatozoa, ova, lipid microparticles, exosomes, micro-organisms, such as worms (C. elegans), plant cells, sub-cellular material such as mitochondria, as well as all manner of biological materials. Hydrogels of the present invention are prepared by combining the hydrogel-forming composition described herein with a cell suspension or other biological sample prior to polymerization. The method may generally comprise the following steps: 1) Mixing a cell suspension with a hydrogel-forming composition described herein, thereby creating a pre-hydrogel polymer solution; 2) applying the compositions described herein to a surface of an imaging container and centrifuging to align cells thereon or allowing them to settle; 3) cross-linking the pre-hydrogel polymer solution by chemical or photo activation to create a polymerized hydrogel; 4) applying reagents, such as fluorescent antibodies, to stain cells and other objects encapsulated within the polymerized hydrogel, and incubating for an appropriate amount of time; 5) removing staining reagents by washing; and 6) evaluating results by imaging.

In a further embodiment, there is provided a method to carrying out repeated immunocytochemistry procedures by photo-bleaching. After encapsulating cells in a polymerized hydrogel, the cells are labeled using reagents, such as fluorescent antibodies, and evaluated by imaging. The locations of the cells are recorded. The sample may then be photo-bleached to render the fluorescent labels inactive. The sample may then be fluorescently labeled again using reagents, such as a different fluorescent antibody or antibodies. The sample may then be evaluated again by imaging. Since the location of the cells are fixed, the signals from multiple labels may be easily attributed to a cell at a particular location within a given imaging container. This procedure could be repeated multiple times to determine signals from many markers simultaneously.

In a further embodiment, there is provided a method of carrying out an automated screening process using the hydrogel-forming compositions and methods described herein. It has been demonstrated that cells can be added to hydrogel-forming compositions described herein, encapsulated therein upon hydrogel polymerization, and then stained using fluorescent compositions. An automated screening process generally comprises of the following steps: 1) dividing the initial cell sample into multiple aliquots, each of which can be stored in a well of a multi-well plate; 2) treating each aliquot with the desired chemical composition and concentration thereof, 3) Adding a hydrogel-forming composition described herein to each aliquot to create a pre-hydrogel polymer solution as described herein; 4) centrifuging the multi-well plate to align the cells at the bottom surface of the well or allowing them to settle; 5) cross-linking the pre-hydrogel polymer solution by chemical or photo activation to create a hydrogel crosslinking the scaffold polymers; 6) applying reagents, such as fluorescent antibodies, to stain cells and other objects within the polymerized hydrogel, and incubating for an appropriate amount of time; 6) removing staining reagents by washing; and 7) evaluating results by imaging.

In an alternative embodiment, a process to evaluate secreted molecules from single cells while phenotyping the cells using immunocytochemistry is provided in FIG. 3, where (A) cells are mixed with the pre-hydrogel polymer solution and added to an imaging container, where the surface of the imaging container is coated with molecules for capturing molecules secreted by the cells and the imaging container may be centrifuged to align the cells to the imaging surface; (B) the pre-hydrogel polymer solution is cross-linked by chemical or photo activation to create a polymerized hydrogel, which spatially constrains the cells; (C) after an appropriate amount of time has elapsed, molecules secreted by each cell are captured by capture molecules surrounding each cell and the pattern of the captured molecules would depend on the amount of secretion; (D) reagents are added to stain both the cell and the captured secreted molecules; and (E) imaging could be used to phenotype each cell, while simultaneously identifying and measuring the amounts of secreted molecules from each cell from the pattern of secreted molecules captured.

In a first embodiment there is provided a composition, the composition including: (a) a scaffold polymer, wherein the scaffold polymer: has one or more acryloyl group or one or more methacryloyl groups; has an average molecular weight (Mₙ) between about 300 and about 6,000; is water soluble and biocompatible; and is operable to form a hydrogel following cross-linking; (b) a porogen; and (c) a crosslinking agent; wherein, the composition has a density of between about 1.0 g/ml and about 1.12 g/ml at 25° C.

The composition may have a density of between about 1.0 g/ml and about 1.11 g/ml at 25° C. The composition may have a density of between about 1.0 g/ml and about 1.10 g/ml at 25° C. The composition may have a density of between about 1.0 g/ml and about 1.09 g/ml at 25° C. The composition may have a density of between about 1.0 g/ml and about 1.08 g/ml at 25° C. The composition may have a density of between about 1.01 g/ml and about 1.10 g/ml at 25° C. The composition may have a density of between about 1.02 g/ml and about 1.08 g/ml at 25° C. The composition may have a density of between about 1.0 g/ml and about 1.07 g/ml at 25° C. The composition may have a density of between about 1.0 g/ml and about 1.06 g/ml at 25° C. The composition may have a density of between about 1.0 g/ml and about 1.05 g/ml at 25° C. The composition may have a density of between about 1.0 g/ml and about 1.04 g/ml at 25° C. The composition may have a density of between about 1.0 g/ml and about 1.067 g/ml at 25° C. The composition may have a density of between about 1.01 g/ml and about 1.067 g/ml at 25° C. The composition may have a density of between about 1.0 g/ml and about 1.066 g/ml at 25° C. The composition may have a density of between about 1.01 g/ml and about 1.066 g/ml at 25° C.

The scaffold polymer may have an average molecular weight (Mₙ) between about 300 and about 3,000. The scaffold polymer may have an average molecular weight (Mₙ) between about 300 and about 2,000. The scaffold polymer may have an average molecular weight (Mₙ) between about 300 and about 1,000. The scaffold polymer may have an average molecular weight (Mₙ) between about 360 and about 3,000. The scaffold polymer may have an average molecular weight (Mₙ) between about 360 and about 2,000. The scaffold polymer may have an average molecular weight (Mₙ) between about 360 and about 1,000. The scaffold polymer may have an average molecular weight (Mₙ) between about 480 and about 3,000. The scaffold polymer may have an average molecular weight (Mₙ) between about 480 and about 2,000. The scaffold polymer may have an average molecular weight (Mₙ) between about 480 and about 1,000. The scaffold polymer may have an average molecular weight (Mₙ) between about 500 and about 3,000. The scaffold polymer may have an average molecular weight (Mₙ) between about 500 and about 2,000. The scaffold polymer may have an average molecular weight (Mₙ) between about 500 and about 1,000. The scaffold polymer may have an average molecular weight (Mₙ) between about 550 and about 3,000. The scaffold polymer may have an average molecular weight (Mₙ) between about 550 and about 2,000. The scaffold polymer may have an average molecular weight (Mₙ) between about 550 and about 1,000. The scaffold polymer may have an average molecular weight (Mₙ) between about 575 and about 3,000. The scaffold polymer may have an average molecular weight (Mₙ) between about 575 and about 2,000. The scaffold polymer may have an average molecular weight (Mₙ) between about 575 and about 1,000. The scaffold polymer may have an average Mₙ between about 300 and about 6,000. The scaffold polymer may have an average Mₙ between about 300 and about 2,000. The scaffold polymer may have an average Mₙ between about 360 and about 2,000. The scaffold polymer may have an average Mₙ between about 400 and about 2,000. The scaffold polymer may have an average Mₙ between about 300 and about 2,000. The scaffold polymer may have an average Mₙ between about 550 and about 2,000. The scaffold polymer may have an average Mₙ between about 575 and about 2,000. The scaffold polymer may have an average Mₙ between about 575 and about 1,000. The scaffold polymer may have an average Mₙ between about 575 and about 700. The scaffold polymer may have an average Mₙ of about 575. The scaffold polymer may have an average Mₙ of about 700. The scaffold polymer may have an average Mₙ of about 1000. The scaffold polymer may have an average Mₙ of about 2000.

The scaffold polymer may be selected from the following: Poly(ethylene glycol) diacrylate (PEGDA); Poly(ethylene glycol) dimethylacrylate (PEGDMA); Poly(ethylene glycol) methyl ether acrylate (PEGMEA); Poly(ethylene glycol) methacrylate (PEGMA); Poly(ethylene glycol) methyl ether methacrylate (PEGMEMA); and Gelatin-methylacrylate (Gelatin-MA). The scaffold polymer may be selected from the following: Poly(ethylene glycol) diacrylate (PEGDA); Poly(ethylene glycol) dimethylacrylate (PEGDMA); Poly(ethylene glycol) methyl ether acrylate (PEGMEA); Poly(ethylene glycol) methacrylate (PEGMA); and Poly(ethylene glycol) methyl ether methacrylate (PEGMEMA). The scaffold polymer may be selected from the following: PEGDA; PEGDMA; PEGMA; and PEGMEMA. The scaffold polymer may be selected from the following: PEGDA and PEGDMA. The scaffold polymer may be PEGDA. The scaffold polymer may be PEGDMA. The scaffold polymer may be PEGMA. The scaffold polymer may be PEGMEA. The scaffold polymer may be PEGMEMA. The scaffold polymer may be Gelatin-MA.

The porogen may be selected from one or more of the following: Poly(ethylene glycol) (PEG); Chitosan; Agarose; Dextran; Hyaluronic acid; Poly(methyl methacrylate) (PMMA); Cellulose and derivatives thereof; Gelatin and derivatives thereof; and Acrylamide and derivatives thereof. The porogen may be selected from the following: Poly(ethylene glycol) (PEG); Chitosan; Agarose; Dextran; Hyaluronic acid; Poly(methyl methacrylate) (PMMA); Cellulose and derivatives thereof; Gelatin and derivatives thereof; and Acrylamide and derivatives thereof. The porogen may be selected from one or more of the following: Poly(ethylene glycol) (PEG); Chitosan; Agarose; Dextran; Hyaluronic acid; Poly(methyl methacrylate) (PMMA); Cellulose and derivatives thereof; and Gelatin and derivatives thereof. The porogen may be selected from one or more of the following: Poly(ethylene glycol) (PEG); Chitosan; Agarose; Dextran; Hyaluronic acid; Poly(methyl methacrylate) (PMMA); and Cellulose and derivatives thereof. The porogen may be selected from one or more of the following: Poly(ethylene glycol) (PEG); Chitosan; Agarose; Dextran; Hyaluronic acid; and Poly(methyl methacrylate) (PMMA). The porogen may be selected from one or more of the following: Poly(ethylene glycol) (PEG); Chitosan; Agarose; Dextran; and Hyaluronic acid. The porogen may be selected from one or more of the following: Poly(ethylene glycol) (PEG); Chitosan; Agarose; and Dextran. The porogen may be selected from one or more of the following: Poly(ethylene glycol) (PEG); Chitosan; and Agarose. The porogen may be selected from one or more of the following: Poly(ethylene glycol) (PEG); and Chitosan. The porogen may be PEG.

The porogen may be PEG and may have an average Mₙ between 8,000 and 40,000. The porogen may be PEG and may have an average Mₙ between 8,000 and 30,000. The porogen may be PEG and may have an average Mₙ between 10,000 and 40,000. The porogen may be PEG and may have an average Mₙ between 10,000 and 30,000. The porogen may be PEG and may have an average Mₙ between 11,000 and 30,000. The porogen may be PEG and may have an average Mₙ between 12,000 and 30,000. The porogen may be PEG and may have an average Mₙ between 13,000 and 30,000. The porogen may be PEG and may have an average Mₙ between 14,000 and 30,000. The porogen may be PEG and may have an average Mₙ between 15,000 and 30,000. The porogen may be PEG and may have an average Mₙ between 16,000 and 30,000. The porogen may be PEG and may have an average Mₙ between 17,000 and 30,000. The porogen may be PEG and may have an average Mₙ between 18,000 and 30,000. The porogen may be PEG and may have an average Mₙ between 19,000 and 30,000. The porogen may be PEG and may have an average Mₙ between 20,000 and 30,000. The porogen may be PEG and may have an average Mₙ between 10,000 and 40,000. The porogen may be PEG and may have an average Mₙ between 11,000 and 40,000. The porogen may be PEG and may have an average Mₙ between 12,000 and 40,000. The porogen may be PEG and may have an average Mₙ between 13,000 and 40,000. The porogen may be PEG and may have an average Mₙ between 14,000 and 40,000. The porogen may be PEG and may have an average Mₙ between 15,000 and 40,000. The porogen may be PEG and may have an average Mₙ between 16,000 and 40,000. The porogen may be PEG and may have an average Mₙ between 17,000 and 40,000. The porogen may be PEG and may have an average Mₙ between 18,000 and 40,000. The porogen may be PEG and may have an average Mₙ between 19,000 and 40,000. The porogen may be PEG and may have an average Mₙ between 20,000 and 40,000. The porogen may be PEG and may have an average Mₙ of 20,000. The porogen may be PEG and may have an average Mₙ between 1,000 and 40,000.

The scaffold polymer may be between 80% w/v and 100% w/v where the average Mₙ is 6,000. The scaffold polymer may be between forms between 30% w/v and 100% w/v where the average Mₙ is 2,000. The scaffold polymer may be between 20% w/v and 100% w/v where the average Mₙ is 1,000. The scaffold polymer may be between 15% w/v and 100% w/v where the average Mₙ is 700. The scaffold polymer may be between 10% w/v and 100% w/v where the average Mₙ is 575. The scaffold polymer may be between 5% w/v and 100% w/v where the average Mₙ is 550. The scaffold polymer may be between 5% w/v and 100% w/v where the average Mₙ is 300.

The composition may have a density less than the cell to be encapsulated.

The proportion of water soluble, biocompatible scaffold polymer to porogen may be >1:2. The proportion of water soluble, biocompatible scaffold polymer to porogen may be ≥1:2. The proportion of water soluble, biocompatible scaffold polymer to porogen may be >1:3. The proportion of water soluble, biocompatible scaffold polymer to porogen may be ≥1:3. The proportion of water soluble, biocompatible scaffold polymer to porogen may be >1:4. The proportion of water soluble, biocompatible scaffold polymer to porogen may be ≥1:4.

The composition may include a weight ratio of the scaffold polymer to porogen may be about 1:1; the scaffold polymer may be PEGDA having an average Mₙ of between about 550 and about 2000 and 15% w/v; and the porogen may be PEG having an average Mₙ of between about 10,000 and about 40,000 and 15% w/v. The composition may include a weight ratio of the scaffold polymer to porogen may be about 1:1; the scaffold polymer may be PEGDA having an average Mₙ of between about 550 and about 2000 and 15% w/v; and the porogen may be PEG having an average Mₙ of 20,000 and 15% w/v. The composition may include a weight ratio of the scaffold polymer to porogen may be about 1:1; the scaffold polymer may be PEGDA having an average Mₙ of 700 and 15% w/v; and the porogen may be PEG having an average Mₙ of 20,000 and 15% w/v.

The weight ratio of the scaffold polymer to porogen may be about 1:1. The scaffold polymer may be PEGDA having an average Mₙ of 700 and 15% w/v. The porogen may be PEG having an average Mₙ of 20,000 and 15% w/v.

The crosslinking agent may be a free-radical generating compound. The crosslinking agent may be biocompatible. The crosslinking agent may be a UV photo-initiator. The crosslinking agent may be a photo-initiator selected from TABLE 1B. The crosslinking agent may be one or more of the photo-initiators selected from TABLE 1B. The crosslinking agent may be Irgacure 819 or Irgacure 2959. The crosslinking agent may be Irgacure 2959. The crosslinking agent may be Irgacure 819.

The crosslinking agent may be Irgacure 2959 at 1.8% w/v or Irgacure 819 at 1.8% w/v. The crosslinking agent may be Irgacure 2959 at 1.0% w/v or Irgacure 819 at 1.0% w/v. The crosslinking agent may be Irgacure 2959 at 0.1% w/v or Irgacure 819 at 0.1% w/v.

In a further embodiment, there is provided a composition, the composition including: (a) a scaffold polymer, wherein the scaffold polymer: is selected from: PEGDA; PEGMA; and PEGDMA; has an average molecular weight (Mₙ) between about 500 and about 3,000; is water soluble and biocompatible; and is operable to form a hydrogel following cross-linking; and (b) 2-Hydroxy-1-[4-(2-hydroxyethoxy)phenyl]-2-methyl-1-propanone is less than or equal to 1.0% w/v of the composition; wherein, the composition has a density of between about 1.0 g/ml and about 1.10 g/ml at 25° C. The composition may further include a porogen. The 2-Hydroxy-1-[4-(2-hydroxyethoxy)phenyl]-2-methyl-1-propanone may be less than or equal to 0.1% w/v of the composition. The 2-Hydroxy-1-[4-(2-hydroxyethoxy)phenyl]-2-methyl-1-propanone may be less than or equal to 0.2% w/v of the composition. The 2-Hydroxy-1-[4-(2-hydroxyethoxy)phenyl]-2-methyl-1-propanone may be less than or equal to 0.3% w/v of the composition. The 2-Hydroxy-1-[4-(2-hydroxyethoxy)phenyl]-2-methyl-1-propanone may be less than or equal to 0.4% w/v of the composition. The 2-Hydroxy-1-[4-(2-hydroxyethoxy)phenyl]-2-methyl-1-propanone may be less than or equal to 0.5% w/v of the composition. The 2-Hydroxy-1-[4-(2-hydroxyethoxy)phenyl]-2-methyl-1-propanone may be less than or equal to 0.6% w/v of the composition. The 2-Hydroxy-1-[4-(2-hydroxyethoxy)phenyl]-2-methyl-1-propanone may be less than or equal to 0.7% w/v of the composition. The 2-Hydroxy-1-[4-(2-hydroxyethoxy)phenyl]-2-methyl-1-propanone may be less than or equal to 0.8% w/v of the composition. The 2-Hydroxy-1-[4-(2-hydroxyethoxy)phenyl]-2-methyl-1-propanone may be less than or equal to 0.9% w/v of the composition.

The 2-Hydroxy-1-[4-(2-hydroxyethoxy)phenyl]-2-methyl-1-propanone may be less than or equal to 0.1% w/v of the composition

In a further embodiment, there is provided a cell encapsulation method, the method including: (a) mixing a composition described herein with a cell or a cell suspension to form a cell polymer mixture; (b) adding the cell polymer mixture to a cell imaging container; (c) settling the cell within the cell imaging container; and (d) cross-linking the cell polymer mixture to form a hydrogel.

In a further embodiment, there is provided a cell encapsulation method, the method including: (a) adding a composition described herein to a cell imaging container; (b) adding a cell or a cell suspension to the cell imaging container onto the composition described herein; (c) settling the cell within the cell imaging container; and (d) cross-linking the cell polymer mixture to form a hydrogel.

The method may further include assaying of the cell or cells encapsulated by the hydrogel using immunocytochemistry. The settling of the cell or cells within the cell imaging container may be by centrifugation. The method may further include bleaching fluorescence and assaying of the cells encapsulated by the hydrogel using immunocytochemistry. The method may further include bleaching the fluorescence from a previous immunocytochemistry assay and assaying of the cells encapsulated by the hydrogel using a second immunocytochemistry assay. This bleaching of a previous immunocytochemistry assay and assaying of the cells encapsulated by the hydrogel using a subsequent immunocytochemistry assay may be repeated as many times as needed. The method may further include repeated bleaching of fluorescence and assaying of the cells encapsulated by the hydrogel using immunocytochemistry.

In a further embodiment, there is provided a cell encapsulation method, the method including: (a) adding a crosslinking agent to the surface of a cell imaging container; (b) adding a composition to the cell imaging container, the composition comprising: (i) a scaffold polymer, wherein the scaffold polymer: has one or more acryloyl group or one or more methacryloyl groups; has an average molecular weight (Mₙ) between about 300 and about 6,000; is water soluble and biocompatible; and is operable to form a hydrogel following cross-linking; and (ii) a porogen; (c) adding cells or a cell suspension to the composition to form a cell polymer mixture in the imaging container; (d) settling the cell within the cell imaging container; and (e) cross-linking the cell polymer mixture to form a hydrogel.

The hydrogel may have a thickness of between about 10 μm and about 1,000 μm. The hydrogel may have a thickness of between about 10 μm and about 900 μm. The hydrogel may have a thickness of between about 10 μm and about 800 μm. The hydrogel may have a thickness of between about 10 μm and about 700 μm. The hydrogel may have a thickness of between about 10 μm and about 600 μm. The hydrogel may have a thickness of between about 10 μm and about 500 μm. The hydrogel may have a thickness of between about 10 μm and about 400 lim. The hydrogel may have a thickness of between about 10 μm and about 300 μm. The hydrogel may have a thickness of between about 10 μm and about 200 μm. The hydrogel may have a thickness of between about 10 μm and about 100 μm.

The hydrogel may have pores between about 10 nm and about 10 μm. The hydrogel may have pores between about 20 nm and about 10 μm. The hydrogel may have pores between about 30 nm and about 10 μm. The hydrogel may have pores between about 40 nm and about 10 lim. The hydrogel may have pores between about 50 nm and about 10 μm. The hydrogel may have pores between about 60 nm and about 10 μm. The hydrogel may have pores between about 70 nm and about 10 μm. The hydrogel may have pores between about 80 nm and about 10 μm. The hydrogel may have pores between about 90 nm and about 10 μm. The hydrogel may have pores between about 100 nm and about 10 μm. The hydrogel may have pores between about 20 nm and about 9 μm. The hydrogel may have pores between about 30 nm and about 9 μm. The hydrogel may have pores between about 40 nm and about 9 μm. The hydrogel may have pores between about 50 nm and about 9 μm. The hydrogel may have pores between about 60 nm and about 9 μm. The hydrogel may have pores between about 70 nm and about 9 μm. The hydrogel may have pores between about 80 nm and about 9 μm. The hydrogel may have pores between about 90 nm and about 9 μm. The hydrogel may have pores between about 100 nm and about 9 μm. The hydrogel may have pores between about 20 nm and about 8 μm. The hydrogel may have pores between about 30 nm and about 8 μm. The hydrogel may have pores between about 40 nm and about 8 μm. The hydrogel may have pores between about 50 nm and about 8 μm. The hydrogel may have pores between about 60 nm and about 8 μm. The hydrogel may have pores between about 70 nm and about 8 μm. The hydrogel may have pores between about 80 nm and about 8 μm. The hydrogel may have pores between about 90 nm and about 8 μm. The hydrogel may have pores between about 100 nm and about 8 μm. The hydrogel may have pores between about 20 nm and about 7 μm. The hydrogel may have pores between about 30 nm and about 7 μm. The hydrogel may have pores between about 40 nm and about 7 μm. The hydrogel may have pores between about 50 nm and about 7 μm. The hydrogel may have pores between about 60 nm and about 7 μm. The hydrogel may have pores between about 70 nm and about 7 μm. The hydrogel may have pores between about 80 nm and about 7 μm. The hydrogel may have pores between about 90 nm and about 7 μm. The hydrogel may have pores between about 100 nm and about 7 μm. The hydrogel may have pores between about 20 nm and about 6 μm. The hydrogel may have pores between about 30 nm and about 6 μm. The hydrogel may have pores between about 40 nm and about 6 μm. The hydrogel may have pores between about 50 nm and about 6 μm. The hydrogel may have pores between about 60 nm and about 6 μm. The hydrogel may have pores between about 70 nm and about 6 μm. The hydrogel may have pores between about 80 nm and about 6 μm. The hydrogel may have pores between about 90 nm and about 6 μm. The hydrogel may have pores between about 100 nm and about 6 μm. The hydrogel may have pores between about 20 nm and about 5 μm. The hydrogel may have pores between about 30 nm and about 5 μm. The hydrogel may have pores between about 40 nm and about 5 μm. The hydrogel may have pores between about 50 nm and about 5 μm. The hydrogel may have pores between about 60 nm and about 5 μm. The hydrogel may have pores between about 70 nm and about 5 μm. The hydrogel may have pores between about 80 nm and about 5 μm. The hydrogel may have pores between about 90 nm and about 5 μm. The hydrogel may have pores between about 100 nm and about 5 μm. The hydrogel may have pores between about 20 nm and about 4 μm. The hydrogel may have pores between about 30 nm and about 4 μm. The hydrogel may have pores between about 40 nm and about 4 μm. The hydrogel may have pores between about 50 nm and about 4 μm. The hydrogel may have pores between about 60 nm and about 4 μm. The hydrogel may have pores between about 70 nm and about 4 μm. The hydrogel may have pores between about 80 nm and about 4 μm. The hydrogel may have pores between about 90 nm and about 4 μm. The hydrogel may have pores between about 100 nm and about 4 μm. The hydrogel may have pores between about 20 nm and about 3 μm. The hydrogel may have pores between about 30 nm and about 3 μm. The hydrogel may have pores between about 40 nm and about 3 μm. The hydrogel may have pores between about 50 nm and about 3 μm. The hydrogel may have pores between about 60 nm and about 3 μm. The hydrogel may have pores between about 70 nm and about 3 μm. The hydrogel may have pores between about 80 nm and about 3 μm. The hydrogel may have pores between about 90 nm and about 3 μm. The hydrogel may have pores between about 100 nm and about 3 μm. The hydrogel may have pores between about 20 nm and about 2 μm. The hydrogel may have pores between about 30 nm and about 2 μm. The hydrogel may have pores between about 40 nm and about 2 μm. The hydrogel may have pores between about 50 nm and about 2 μm. The hydrogel may have pores between about 60 nm and about 2 μm. The hydrogel may have pores between about 70 nm and about 2 μm. The hydrogel may have pores between about 80 nm and about 2 μm. The hydrogel may have pores between about 90 nm and about 2 μm. The hydrogel may have pores between about 100 nm and about 2 μm. The hydrogel may have pores between about 20 nm and about 1 μm. The hydrogel may have pores between about 30 nm and about 1 μm. The hydrogel may have pores between about 40 nm and about 1 μm. The hydrogel may have pores between about 50 nm and about 1 μm. The hydrogel may have pores between about 60 nm and about 1 μm. The hydrogel may have pores between about 70 nm and about 1 μm. The hydrogel may have pores between about 80 nm and about 1 μm. The hydrogel may have pores between about 90 nm and about 1 μm. The hydrogel may have pores between about 100 nm and about 1 μm.

The cross-linking may be by UV light. The cross-linking may be by UV light at a wavelength between about 300 nm and about 375 nm. The cross-linking may be by UV light at a wavelength between about 300 nm and about 375 nm for an exposure of 5 seconds or less. In a further embodiment, there is provided a cell encapsulation kit, the kit including: a composition described herein; and instructions for the compositions use in the encapsulation of cells.

The kit may further include immunocytochemistry reagents. The kit may further include an imaging container.

### BRIEF DESCRIPTION OF THE DRAWINGS

FIG. 1A: shows a schematic workflow to prepare cells for immunocytochemistry (ICC) using a polymer hydrogel encapsulation: in 100 the PEGDA pre-hydrogel polymer solution and cell suspension, with individual cell (101) is added to an imaging well-plate, where the plate is optionally centrifuged to settle the cells (101) within the pre-hydrogel polymer solution (102) at the bottom of the plate (but may be allowed to settle without centrifugation) and the plate is exposed to UV light (103); in 200 supernatant, along with uncured pre-hydrogel polymer solution (203) is removed from the well via pipette (204) leaving the cross-linked hydrogel (202) with encapsulated cells (201); in 300 conventional immunostaining is being carried out on the cells (301) within the cross-linked hydrogel (302), which may include cell fixation, permeabilization, intracellular and surface antibody staining, as well as the multiple washing steps (or ICC reagents (303)) using a pipette (304) and may be carried out in the well, without additional centrifugation steps; and in 400 image acquisition can be performed directly on the imaging plate, where stained cells (401) may be viewed with a microscope objective lens (404) within the cross-linked hydrogel (402), with or without buffer solution (403).

FIG. 1B: shows a schematic close-up of cells encapsulated in the hydrogel matrix within a single well of an imaging container (503), and a series of cut-out magnified views of a portion of the cells: in 500 the cells (501) are shown in the pre-hydrogel polymer solution (502); in 600 the cut-out magnified view now contains cells (601) are shown in the cross-linked hydrogel matrix (602), showing the uncured porogen polymer (603); in 700 the same cross-linked hydrogel matrix (702) is shown encapsulating the cells (701) and with pores (703) following removal of the porogen; and in 800 shows the same cross-linked hydrogel matrix (802) encapsulating cells (801) with antibodies (804) able to access the cells (801) via the pores (803). The antibodies may be tagged in some way to facilitate visualization using ICC techniques.

FIG. 2: shows a comparison of cell loss using different ICC reagents and procedures. Results are shown as mean±standard deviation (SD) of manual cell counts. The PEGDA cell encapsulation process showed 1-3% cell loss for all cell dilutions, which was considered as error from manual count. The standard and Cytospin™ methods showed more than 50% cell loss for all cell concentrations and 100% cell loss when 10 or less cells were spun onto the slide.

FIG. 3: shows the process to evaluate secreted molecules from single cells while phenotyping the cells using immunocytochemistry. (A) Cells are mixed with the pre-hydrogel polymer solution and added to an imaging container. The surface of the imaging container is coated with molecules for capturing molecules secreted by the cells. The imaging container is centrifuged to align the cells to the imaging surface. (B) The pre-hydrogel polymer solution is cross-linked by chemical or photo activation to create a polymerized hydrogel, which spatially constrains the cells. (C) After an appropriate amount of time has elapsed, molecules secreted by each cell are captured by capture molecules surrounding each cell. The pattern of the captured molecules would depend on the amount of secretion. (D) Reagents are added to stain both the cell and the captured secreted molecules. (E) Imaging could be used to phenotype each cell, while simultaneously identifying and measuring the amounts of secreted molecules from each cell from the pattern of secreted molecules captured.

### DETAILED DESCRIPTION OF THE INVENTION

Any terms not specifically defined herein shall be understood to have the meanings commonly associated with them as understood within the art of the invention.

### Definitions

“Polymerization” is defined herein as a process of reacting monomer molecules together in a chemical reaction to form polymer chains.

“Cross-linking agent” is defined herein as a bond or bonds that link one polymer chain to another via covalent bonds or ionic bonds. In the case of scaffold polymers having one or more acryloyl groups or one or more methacryloyl groups, the cross-linking would occur between the scaffold polymer chains at their acryloyl or methacryloyl termini, in the presence of a cross-linking agent and upon exposure to ultraviolet (UV) light.

A “biocompatible” is defined herein as any composition component that has limited or no cytotoxicity at the concentration it is being used.

Free-radical polymerization (FRP) is a method of polymerization by which a polymer forms by the successive addition of free-radical building blocks. Free radicals can be formed by a number of different mechanisms, usually involving separate initiator molecules. Following its generation, the initiating free radical adds (non-radical) monomer units, thereby growing the polymer chain.

A photo-initiator is a type of crosslinking agent that creates a reactive species (free radicals, cations or anions) when exposed to radiation (UV or visible). A number of possible photo-initiators are described in TABLE 1B and may be selected based on the particular immunocytochemistry use anticipated for the cell encapsulation hydrogel and to work well with the particular scaffold polymer chosen and the detectable tag or tags being utilized.

“Ultra-violet cross-linking” is defined herein as the use of ultra-violet (UV) radiation to create reactive species (free radicals, cations or anions) upon exposure to UV radiation. The process may be assisted by the presence of a photo-initiator. Where crosslinking is done with UV, the ability to cure a polymer composition described herein (i.e. scaffold polymer, crosslinking agent and/or porogen) into a hydrogel improves with decreasing wavelength. Whereby most of the hydrogels formed were at 375 nm UV for usually no more than a 5 minute exposure with 0.1% 2959 Irgacure™. However, where a composition does not cure well using these parameters, the wavelength of the UV can be reduced to 365 nm, 355 nm, 345 nm, 335 nm, 325 nm, 315 nm and 305 nm to increase curing of the hydrogel. Furthermore, the reduction in wavelength (although making the UV more difficult to use due to safety considerations) would penetrate the pre-hydrogel polymer solution and thus be more effective at crosslinking the scaffold polymers. Below 300 nm, the absorption of glass starts to increase, but how much UV light is lost to glass depends on glass thickness, which is very thin (˜170 urn) for an imaging micro-well plate. Cell viability is not a concern where the cells are fixed and permeabilized, but when a viable cell is needed for an ICC assay or there is a wish to recover live cells then UV wavelength used to cure the hydrogel becomes more important UV light below 300 nm will begin to be absorbed by DNA, RNA, and proteins. Under low wavelength UV light peptide bonds may come lose, which will degrade the sample. Without changing the wavelength, the amount of photo-initiator may also be increased to improve curing time and the ability to cure. For example, in going from 0.1% to 1.0% 2959 Irgacure™ reduced curing time and curability of a pre-hydrogel polymer solution. However, this increase in photo-initiator concentration can have negative effects on cell viability and increased background fluorescence of the resulting hydrogel.

“Immunocytochemistry” (ICC) is defined herein as a method of direct or indirect anatomical visualization of the localization of a specific protein or antigen in cells by use of one or more specific antibodies that bind to cell features of interest (i.e. proteins or other molecules within or on cell—antigens). The antibodies may have a detectable tag attached (direct visualization) or a detectable tag may be attached to a secondary antibody that binds to a primary antibody (indirect visualization). The primary antibody or antibodies allow for the visualization of the cell feature under microscope (for example, a fluorescence microscope, confocal microscope or light microscope) when bound by a secondary antibody or an antibody with a detectable tag attached. Immunocytochemistry allows for an evaluation of whether or not cells in a particular sample express the antigen, where on or in a cell the immune-positive signal may be found and the relative quantities of those antigens.

ICC is a biological technique for assaying cells in both research and diagnostic applications. However, standard ICC methods often do not work well when the cell sample contains a small number of cells (<10,000) because of the significant cell loss that occurs during washing, staining, and centrifugation steps. Such losses are also a significant problem when working with rare cells, such as circulating tumor cells, where losses could significantly bias experimental outcomes.

A “detectable tag” as defined herein refers to any moiety that may be attached directly to an antibody that is then allowed to bind to an antigen or to another antibody already bound to the antigen in a cell. Antibodies may be labeled with small molecules, radioisotopes, gold particles, enzymatic proteins, fluorescent dyes, fluorescent molecules, chromogenic molecules or combinations thereof. The particular detectable tag will depend on the ICC method or methods being carried out.

For example, biotin-labeled antibodies may be followed by a second incubation with avidin or streptavidin, where the avidin or streptavidin is labeled with an enzyme or a fluorescent dye. Antibodies are often conjugated with multiple biotin molecules (3-6 molecules), which may lead to an amplification step that enhances detection of less abundant antigens.

Fluorescent tags may be covalently attached to antibodies through primary amines or thiol groups. Fluorescently-labeled antibodies can be purchased from many companies, or commercial kits are available for labeling of antibodies in the lab. To detect a fluorescent label, an instrument is required that emits a specified wavelength of light that excites the fluorochrome. The fluorescent dye then emits a signal in a different wavelength. The same instrument contains appropriate filters for detecting the emission from the fluorochrome. Antibodies can be labeled with a variety of fluorescent dyes with varying excitation and emission spectra. In addition to being highly quantitative, fluorescent labels give the distinct advantage of being able to multiplex, or detect two or more different target proteins at the same time, through the use of dyes with non-overlapping emission spectra.

A “polymer” is defined herein as any large molecule, or macromolecule, made up of many repeated subunits, (for example, polysaccharides or polypeptides). Polymers may be synthetic (for example, PEGDA, PEGMA, PEGMEA, PEGDMA or PEGMEMA) or may be naturally occurring biological macromolecules (for example, polysaccharides like carrageenan, agarose/agar, chitosan and gelatin).

A “scaffold polymer” is defined herein as a specific subgroup of polymers having very particular characteristics that make them suitable for use in cell encapsulation in a hydrogel for use in ICC. The particular characteristics of the scaffold polymers that are significant in choosing an appropriate scaffold polymer are as follows:
(A) have one or more acryloyl group or one or more methacryloyl groups;
(B) have an average molecular weight (Mₙ) between about 300 and about 6,000;
(C) have a density less than the cell to be encapsulated (for example, 1.12-1.09 g/ml for erythrocytes⁴⁴; peripheral blood mononuclear cells (PBMCs) density is between about 1.067 to about 1.077 g/ml⁴³; 1.07-1.10 g/ml for hepatocytes; 1.06 g/ml skeletal muscle; and 1.069-1.096 g/ml fibroblasts, where measured at 25° C.);
(D) is water soluble and biocompatible; and
(E) is at a % w/v of the overall composition such that the polymer is able to crosslink to other polymers and have sufficient mechanical stability to withstand at least 10 or more pipettings of 80 μl/s of 40 μls of PBS through a 200 μl pipette tip (with an opening bore of 460 μm) without significant structural disintegration (i.e. cracks, tears, delamination of the thin layer hydrogel formed after crosslinking).

As used herein “mechanical stability” refers to the ability of a hydrogel to withstand pipettings of 40 ills of PBS at 80 μl/s through a 200 μl pipette tip (with an opening bore of 460 urn) without significant structural disintegration (i.e. cracks, tears, delamination of the thin layer hydrogel formed after crosslinking). A lower limit of at least 10 pipettings of 40 μls of PBS at 80 μl/s through a 200 μl pipette tip (with an opening bore of 460 μm) was determined as a useful lower limit in order to carry out some basic ICC evaluation of a cell. However, if multiple washes and re-staining of the encapsulated cells is anticipated, then a higher mechanical stability may be needed.

Alternatively, lowering the flow rate or increasing the pipette bore could reduce the mechanical strain when manipulating ICC solutions adjacent to the hydrogel. Depending on the scaffold polymer being used, the % w/v of scaffold polymer of the overall composition, the crosslinking agent or photo-initiator selected, the % of crosslinking agent or photo-initiator, the length time the composition is exposed to UV light and the wavelength of that light may all be factors in determining the scaffold polymer's ability to crosslink to other scaffold polymers and the subsequent mechanical stability and thickness and swelling of the resulting hydrogel. Alternative methods for analyzing hydrogel mechanical stability are known in the art^{41, 42, 45}.

The scaffold polymer may be a derivative of polyethylene glycol (PEG) as shown in TABLE 1A, PEG diacrylate (PEGDA); PEG dimethylacrylate (PEGDMA); PEG methyl ether acrylate (PEGMEA); PEG methacrylate (PEGMA); or Poly(ethylene glycol) methyl ether methacrylate (PEGMEMA). Alternatively, the scaffold polymer may be a naturally occurring biological macromolecule (for example, polysaccharides like carrageenan, agarose/agar, chitosan, gelatin and gelatin-methylacrylate (gelatin-MA). Alternatively, the scaffold polymer may be poly(methyl methacrylate) (PMMA), hyaluronic acid, hydroxyethyl methacrylate (HEMA), or N-(2-hydroxypropyl) methacrylamide (HPMA). The scaffold polymer may be a PEGDA with an average Mₙ in the range of about 575 Da-6,000 Da. The scaffold polymer may be a modified PEG with an average Mₙ in the range of about 300 Da-6,000 Da. The scaffold polymer may be a modified PEG with an average Mₙ in the range of about 360 Da-3,000 Da. The scaffold polymer may be a modified PEG with an average Mₙ in the range of about 360 Da-2,000 Da. The scaffold polymer may be PEGDA 700. Alternatively, the scaffold polymers may be four arm or multi-arm polymers and not just the linear polymers shown in TABLE 1A.

An acryloyl or methacryloyl are unsaturated carbonyl compounds having a carbon-carbon double bond and a carbon-oxygen double bond in close proximity (see TABLE 1A), which permits these groups to readily participate in radical-catalysed polymerization at the C═C double bond. Scaffold polymers having carbon-carbon double bonds (for example, Poly(ethylene glycol) diacrylate (PEGDA); Poly(ethylene glycol) dimethylacrylate (PEGDMA); Poly(ethylene glycol) methyl ether acrylate (PEGMEA); Poly(ethylene glycol) methacrylate (PEGMA); and Poly(ethylene glycol) methyl ether methacrylate (PEGMEMA)), are able to readily form high-molecular-weight kinetic chains, wherein the carbon-carbon double bonds serve as crosslinking points. Some commercially available modified PEG polymers have variability in the degree to which termini are modified and this may account for variability in the ability of the scaffold polymers to cross-link to one another and could result in reduced mechanical stability or even inability to cure into a hydrogel. Alternatively, additional co-polymers could be used to facilitate cross-linking and hydrogel formation. It was also observed the methacryloyl PEG polymers had greater hydrogel swelling than PEG polymers with acryloyl termini. The resulting swelling can result in delamination from the glass imaging surface.

TABLE 1A
Polyethylene Glycol (PEG) Scaffold Polymers with Acryloyl or Methacryloyl Groups

| Polyethylene Glycol (PEG) Scaffold Polymers | |
|---|---|
| with Acryloyl or Methacryloyl Groups | Structure |
| Poly(ethylene glycol) diacrylate (PEGDA) | 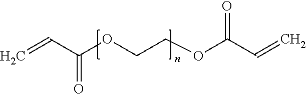 |
| Poly(ethylene glycol) dimethylacrylate (PEGDMA) | 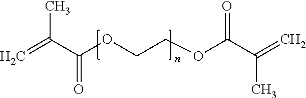 |
| Poly(ethylene glycol) methacrylate (PEGMA) | 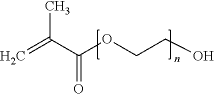 |
| Poly(ethylene glycol) methyl ether acrylate (PEGMEA) | 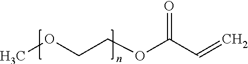 |
| Poly(ethylene glycol) methyl ether methacrylate (PEGMEMA) | 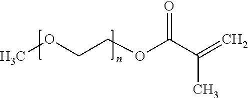 |

A “porogen” is defined herein as a second polymer that may be mixed with the scaffold polymer (first polymer) such that the porogen forms pores when a scaffold polymer is polymerized to form a hydrogel and the porogen is removed. The porogen may be chosen in such a way as to produce hydrogel pores having a defined pore volume, pore size within a hydrogel. The pore size suitable for ICC should be sufficient to allow the transit of staining reagents, with antibodies or fragments thereof as the largest molecule. Antibodies are typically 10 nm to 15 nm across their widest dimension, but the actual size depends on charge, which would depend on the media in which they are found. Pore sizes may also be up to a size that would prevent the release of the cell being encapsulated from the hydrogel during ICC washings. Generally, the range of pore sizes may be between 10 nm and 10 urn. A porogen ideally would not significantly form crosslinks with the scaffold polymer and could thus be removed from the hydrogel following crosslinking to leave pores suitable for ICC.

The porogen may be PEG and/or derivatives of PEG, chitosan, agarose, dextran, hyaluronic acid, PMMA, cellulose and/or cellulose derivatives, gelatin and/or gelatin derivatives, acrylamide and/or acrylamide derivatives, provided that the porogen chosen does not significantly crosslink to the scaffold polymer or cell. The cellulose derivatives may for example be methylcellulose and nitrocellulose. In one embodiment, the porogen is PEG. In another embodiment, the porogen is a PEG derivative. In a further embodiment, the porogen is PEG with a molecular weight >1,000 Da. Alternatively, the porogen is PEG 20,000.

Pores in a hydrogel may be created without the use of a porogen, where the scaffold polymer selected for (a) a higher average Mn; (b) is selected to achieve a lower % w/v of the overall composition; (c) the UV exposure time is adjusted; or (d) a combination of (a), (b) and (c), provided that the hydrogel is able to cure and has sufficient mechanical stability as described herein.

The pore sizes of the hydrogels may be in the range of about 10 nm-10 urn. In another embodiment, the pore sizes may be in the range of about 10 nm-1 μm. Pore sizes can be modulated by a number of factors including, for example, concentration of cross-linking agent, time and intensity of light exposure, molecular weight of scaffold polymer, molecular weight of porogen, ratio of scaffold polymer to porogen. The porous hydrogels of the present invention allow diffusion of certain substances while acting as a mechanical barrier to others. In this way, encapsulation of cells within the hydrogel can reduce cell loss while permitting transmission of antibodies across the hydrogel, for example. Thus, the hydrogels of the present invention are useful in performing immunocytochemical-staining procedures.

The proportion of the water soluble, biocompatible scaffold polymer to porogen may be where the 0.1% Irgacure 2959 and 375 nm UV in order to cure a hydrogel. However, this it is possible to cure with <15% scaffold or <1:2 scaffold:porogen where a lower wavelength UV and/or higher concentration of photo-initiator is used, but the mechanical stability will also in some circumstances also be degraded.

Alternatively, the pores may be generated in the absence of a porogen. For example, the cells could be visualized prior to cross-linking a mask may be created wherein the mask was smaller than the cells (i.e. 10 nm-10 μm), but centered on the cell to prevent polymerization with UV light and to create a pore to each of the cells⁴⁶.

Hydrogel polymerization can be initiated using an appropriate crosslinking agent or photo-initiator. The crosslinking agent may be chemically-activated, which initiates crosslinking upon contact Chemically-activated crosslinking agents may include but are not limited to, acetyl acetone peroxide, acetyl benzoyl peroxide, ascaridole, and tert-butyl hydroperoxide. Alternatively, the crosslinking agent may be photo-activated, which initiates crosslinking after exposure to UV and/or visible light. Examples of photo-activated crosslinking agents (or photo-initiators) may include but are not limited to those found in TABLE 1B. Alternatively, the photo-initiator may be selected from one or more of 2-Hydroxy-4′-(2-hydroxyethoxy)-2-methylpropiophenone (i.e. Irgacure™ 2959), Bis(2,4,6-trimethylbenzoyl)-phenylphosphineoxide (or Irgacure™ 819), 2,2-dimethoxy-2-phenylacetophenone (or DMPA™), Isopropylthioxanthone (or ITV™) or lithium phenyl-2,4,6-trimethylbenzoylphosphinate (LAP™). The photo-initiator may be Irgacure™ 2959. As described herein the photo-initiator or cross-linking agent may be selected based on the desired use for the hydrogel. For example, Irgacure™ 819 and LAP™ makes hydrogel cross-linking (i.e. curing) easier, but result in greater auto-fluorescence when compared with Irgacure™ 2959.

TABLE 1B
Exemplary Photo-initiators

| | | | UV/Visible Light |
|---|---|---|---|
| | | | Absorption |
| | | | Peaks (nm) in |
| Photo-initiator | Chemical Name | Structure | methanol |
| IRGACURE ™ 184 | 1-Hydroxy-cyclohexyl- phenyl-ketone | 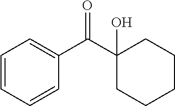 [SMILES: O=C(c1ccccc1)C1(O)CCCCC1] | 246, 280, 333 |
| IRGACURE ™ 500 | IRGACURE 184 (50 wt %) | | 250, 332 |
| | Benzophenone (50 wt %) | | |
| | (DAROCUR BP) | | |
| DAROCUR ™ 1173 | 2-Hydroxy-2-methyl-1- phenyl-1-propanone | 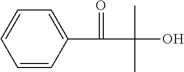 | 245, 280, 331 |
| IRGACURE ™ 2959 | 2-Hydroxy-1-[4-(2- hydroxyethoxy)phenyl]-2- methyl-1-propanone | 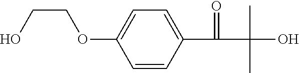 [SMILES: CC(C)(O)C(=O)c1ccc(OCCO)cc1] | 276 |
| DAROCUR ™ MBF | Methylbenzoylformate | 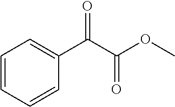 | 255, 325 |
| IRGACURE ™ 754 | oxy-phenyl-acetic acid 2-[2 | | 255, 325 |
| | oxo-2phenyl-acetoxy- | | |
| | ethoxy]-ethyl ester and oxy- | | |
| | phenyl-acetic 2-[2-hydroxy- | | |
| | ethoxy]-ethyl ester | | |
| IRGACURE ™ 651 | Alpha, alpha-dimethoxy- alpha-phenylacetophenone | 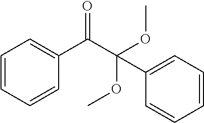 [SMILES: COC(OC)(C(=O)c1ccccc1)c1ccccc1] | 250, 340 |
| IRGACURE ™ 369 | 2-Benzyl-2-(dimethylamino)- 1-[4-(4-morpholinyl) phenyl]-1-butanone | 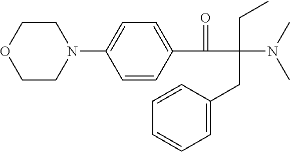 [SMILES: CCC(Cc1ccccc1)(C(=O)c1ccc(N2CCOCC2)cc1)N(C)C] | 233, 324 |
| IRGACURE ™ 907 | 2-Methyl-1-[4- (methylthio)phenyl]-2-(4- morpholinyl)-1-propanone | 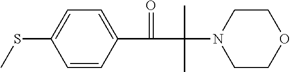 [SMILES: CSc1ccc(C(=O)C(C)(C)N2CCOCC2)cc1] | 230, 304 |
| IRGACURE ™ 1300 | IRGACURE 369 (30 wt %) | | 251,323 |
| | IRGACURE 651 (70 wt %) | | |
| DAROCURE ™ TPO | Diphenyl (2,4,6- trimethylbenzoyl) phosphine oxide | 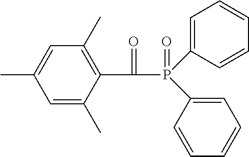 [SMILES: Cc1cc(C)c(C(=O)P(=O)(c2ccccc2)c2ccccc2)c(C)c1] | 295, 368, 380, 393 |
| DAROCUR ™ 4265 | DAROCUR TPO (50 wt %) | | 240, 272, 380 |
| | DAROCUR 1173 (50 wt %) | | |
| IRGACURE ™ 819 | Phosphine oxide, phenyl bis(2,4,6-trimethyl benzoyl) | 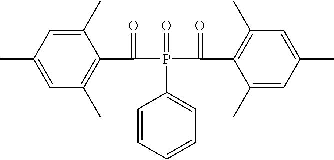 | 295, 370 |
| IRGACURE ™ | IRGACURE 819 (45% active) | | 295, 370 |
| 819DW | dispersed in water | | |
| IRGACURE ™ 2022 | IRGACURE 819 (20 wt %) | | 246, 282, 370 |
| | DAROCUR 1173 (80 wt %) | | |
| IRGACURE ™ 2100 | | | 275, 370 |
| IRGACURE ™ 784 | Bis (eta 5-2,4- cyclopentadien-1-yl) Bis [2,6- difluoro-3-(1H-pyrrol-1- yl)phenyl]titanium | 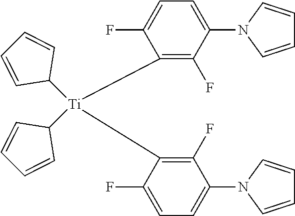 | 398, 470 |
| IRGACURE ™ 250 | Iodonium, (4- methylphenyl)[4-(2- methylpropyl) phenyl- hexafluorophosphate (1-) | 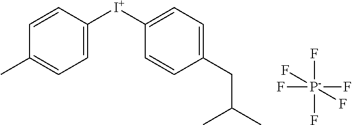 [SMILES: Cc1ccc([I+]c2ccc(CC(C)C)cc2)cc1 ; F[P-](F)(F)(F)(F)F] | 242 |
| DAROCUR ™ BP | Benzophenone | 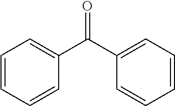 [SMILES: O=C(c1ccccc1)c1ccccc1] | |
| DMPA | 2,2-dimethoxy-2- phenylacetophenone | 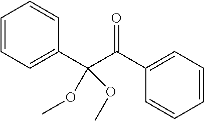 [SMILES: COC(OC)(C(=O)c1ccccc1)c1ccccc1] | |
| ITX | Isopropylthioxanthone | 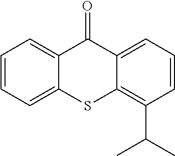 [SMILES: CC(C)c1cccc2c(=O)c3ccccc3sc12] | |
| LAP | lithium phenyl-2,4,6- trimethylbenzolphosphinate | 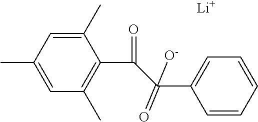 | |

DAROCUR ™ and IRGACURE ™ are made by Ciba Specialty Chemicals, Tarrytown, NY

In one embodiment, the density of the pre-hydrogel polymer solution is greater than the density of the solvent and less than the density of the encapsulated cells. For most mammalian cells, the preferred density of the cell encapsulation polymer prior to cross-linking is between about 1.0 g/ml and about 1.12 g/ml at 25° C. or alternatively the cell encapsulation polymer prior to cross-linking would have a density of between about 1.0 g/ml and about 1.08 g/ml at 25° C. (see TABLE 2A and 2B). The solvent may be water, PBS, Tris-EDTA (TE) buffer, Tris-acetate-EDTA (TAE) buffer, different types of cell culture media, various staining buffers. In one embodiment, the hydrogel-encapsulated cells can be applied to a surface of an imaging container by for example, centrifugation, thereby forming a film of encapsulated cells thereon. The imaging container may be a slide, a coverslip, an imaging well plate, a microtiter plate, etc. The hydrogel film may have a thickness in the range of about 10 μm-1000 μm.

Most cells have a density in the range of 1.03 g/ml and 1.2 g/ml (for example, 1.12-1.09 g/ml for erythrocytes⁴⁴; peripheral blood mononuclear cells (PBMCs) density is between about 1.067 to about 1.077 g/ml⁴³; 1.07-1.10 g/ml for hepatocytes; 1.06 g/ml skeletal muscle; and 1.069-1.096 g/ml fibroblasts). Thus, compositions for cell encapsulation described herein could be designed to ensure that their density is less than that of the cell or cells to be encapsulated. However, for cells having densities less than or equal to 1.0 g/ml (for example, adipocyte cells—0.92 g/ml), the cells could be attached to the surface of the imaging container prior to encapsulation. Alternatively, bacteria, viruses, or other non-human cells may be encapsulated. Methods for cell density measurements are well known in the art⁴⁴.

Human peripheral blood mononuclear cells (PBMCs) are isolated from peripheral blood and identified as any blood cell with a round nucleus (for example, lymphocytes, monocytes, T-cells (for example, CD3⁺, CD4⁺ and CD8⁺), B-cells, natural killer cells (NK cells), dendritic cells and stem cells). The cell fraction corresponding to red blood cells and granulocytes (neutrophils, basophils and eosinophils) may be separated from whole blood by density gradient centrifugation. A gradient medium may be used (usually of density of 1.077 g/ml) to create a red blood cell and PMN fraction (higher density-lower fraction) and a PBMC fraction (low density-upper fraction). Protocols for such gradient isolation of PMBCs are well known in the art (Böyum A. Scand J Clin Lab Invest Suppl. (1968) 97:77-89 “Isolation of mononuclear cells and granulocytes from human blood. Isolation of mononuclear cells by one centrifugation, and of granulocytes by combining centrifugation and sedimentation at 1 g”). PBMCs originate from hematopoietic stem cells (HSCs) in the bone marrow and give rise to all blood cells of the immune system and HSCs progress through hematopoiesis to produce myeloid and lymphoid cell lineages.

TABLE 2A
Polymer Densities for a Variety of Biocompatible Scaffold Polymers Having One or More
Acryloyl or Methacryloyl Groups

| Polymer | Density at 25° C. | | CAS # (Sigma-Aldrich Catalogue #) |
|---|---|---|---|
| PEGDA average Mₙ 250 | 1.11 | g/mL | 26570-48-9 (475629) |
| (water insoluble) | | | |
| PEGDA average Mₙ 575 | 1.12 | g/mL | 26570-48-9 (437441) |
| PEGDA average Mₙ 700 | 1.12 | g/mL | 26570-48-9 (455008) |
| PEGDA average Mₙ 1000 | 1.12 | g/mL | 26570-48-9 (729086) |
| PEGDA average Mₙ 2000 | 1.12 | g/mL | 26570-48-9 (701971) |
| PEGDA average Mₙ 6000 | 1.12 | g/mL | 26570-48-9 (701963) |
| PEGDA average Mₙ 10000 | 1.12 | g/mL | 26570-48-9 (729094) |
| PEGDA average Mₙ 20000 | 1.12 | g/mL | 26570-48-9 (767549) |
| PEGDMA average Mₙ 550 | 1.099 | g/mL | 25852-47-5 (409510) |
| PEGDMA average Mₙ 750 | 1.11 | g/mL | 25852-47-5 (437468) |
| PEGDMA average Mₙ 2000 | 1.11 | g/mL | 25852-47-5 (687529) |
| PEGDMA average Mₙ 6000 | 1.11 | g/mL | 25852-47-5 (687537) |
| PEGDMA average Mₙ 20000 | 1.11 | g/mL | 25852-47-5 (725692) |
| PEGMA average Mₙ 360 | 1.105 | g/mL | 25736-86-1 (409537) |
| PEGMA average Mₙ 500 | 1.101 | g/mL | 25736-86-1 (409529) |
| PEGMEA average Mₙ 480 | 1.09 | g/mL | 32171-39-4 (454990) |
| PEGMEA average Mₙ 2000 | 1.09 | g/mL | 32171-39-4 (730270) |
| PEGMEMA average Mₙ 300 | 1.05 | g/mL | 26915-72-0 (447935) |
| PEGMEMA average Mₙ 500 | 1.08 | g/mL | 26915-72-0 (447943) |
| PEGMEMA average Mₙ 950 | 1.1 | g/mL | 26915-72-0 (447951) |
| PEGMEMA average Mₙ 1500 | 1.100 | g/cm³ | 26915-72-0 (730319) |
| PEGMEMA average Mₙ 4000 | 1.100 | g/cm³ | 26915-72-0 (730327) |
| Gelatin methacryloyl | 1.2 | g/mL | (900496) |

Numerous possible scaffold polymers were considered herein and are represented in TABLE 2B below.

TABLE 2B
Possible Scaffold Polymers Sorted based on Density

| Polymer | Aqueous | Density | Polymerization |
|---|---|---|---|
| PEGDA | Y (MW > 250) | 1.12 | UV |
| PEGMA | Y (MW > 250) | 1.1 | UV |
| PEGMEA | Y (MW > 250) | 1.09 | UV |
| PEGDMA | Y (MW > 250) | 1.11 | UV |
| PEGMEMA | Y (MW > 250) | 1.05-1.1 | UV |
| Poly(N-isopropylacrylamide) | Y | 1.1 | Cool |
| PMMA | N | 1.18 | UV |
| 2-hydroxyethyl methacrylate | Y | 1.073 | UV (radical) |
| (HEMA) | | | |
| N-(2-Hydroxypropyl) | Y | 1.002 | Need co-polymer |
| methacrylamide (HPMA) | | | |
| Hyaluronic acid | Y | 1.8 | Need co-polymer |
| PVA | Y | 1.19 | Cool |
| PAA | Y | 1.15 | Cool |
| Gelatin | Y | 1.20 | Cool |
| Gelatin-MA | Y | 1.20 | UV |
| Methylcellulose | Y | 1.31 | Heat |
| Carrageenan | Y | 1.37 | Cool |
| Carrageenan-MA | Y | 1.37 | UV |
| Pectin | Y | 1.515 | Cool |
| Agarose/Agar | Y | 1.64 | Cool |
| Agarose-MA | Y | 1.64 | UV |
| Chitin | N | | |
| Chitosan | Y[PH < 6.5] | | Ionic |
| Chitosan-glycol-MA | Y | | UV |

As shown in TABLE 2B above, PMMA and Chitin would not be suitable scaffold polymers since they are not water soluble. Similarly, Chitin and Chitosan would not be suitable scaffold polymers, since they only dissolve in acidic media (for example, Chitosan needs a pH<6.5). Poly(N-isopropylacrylamide) would be a less than ideal scaffold polymer since a hydrogel can easily be reversed at relatively low temperature (32° C.) and has insufficient permeability. HPMA requires co-polymers for cross-linking and the properties vary depending on co-polymer that are used, which makes HPMA hard control during the cross-linking process and thus would make it difficult to control the resulting hydrogel thickness. Hyaluronic acid would be a less than ideal scaffold polymer due to the relatively high density and requires a co-polymer for cross-linking. Pectin, carrageenan and agarose would be less than ideal scaffold polymers since the permeability of these polymers is very small and would likely be incompatible for use with porogen, due to the high degree of phase separation when used with a porogen. Also, the densities of pectin, carrageenan, agarose are too high and thus not permeable enough. Methylcellulose is not suitable since heat is needed to maintain the gel form, which would be detrimental to cell viability and the permeability of methylcellulose is very small. PVA, PVA/PAA would be a less than ideal scaffold polymers since they are incompatible with porogen due to a high degree of phase separation during cross-linking.

It has been demonstrated that cells can be added to hydrogel-forming compositions as described herein and encapsulated therein upon hydrogel formation by cross-linking of scaffold polymers to mechanically constrain the cells within the hydrogel. Molecules secreted by the cells, such as antibodies and cytokines, can be captured using capture molecules immobilized to a container surface, and later detected using detection molecules (ex. fluorescently labeled detection molecules). A hydrogel as described herein may therefore reduce the diffusion of cell-secreted molecules and constrain their capture near each source cell. After capturing the cell-secreted molecules, detection molecules could be used to detect the cell-secreted molecules, while simultaneously performing immunocytochemistry to phenotype the hydrogel-encapsulated cells. The magnitude and spatial pattern of the secreted molecules can be detected by imaging to measure the identity and amounts of secreted molecules released from each cell. The ability to simultaneously measure secreted molecules and phenotype single cells overcomes a key challenge in existing ELISpot assays, which can detect secreted molecules from single cells, but cannot simultaneously phenotype the cells. Whereas flow cytometry assays can phenotype single cells, but cannot simultaneously measure secretion.

In further embodiment, there is provided a method of carrying out immunocytochemistry while simultaneously evaluating secreted molecules from single cells using the hydrogel-forming compositions and methods described herein. The method generally comprising the following steps: 1) Mixing a cell suspension with a hydrogel-forming composition described herein to create a pre-hydrogel polymer solution; 2) Applying the pre-hydrogel polymer solution to an imaging container, the surface of which, has been coated with chemicals to capture molecules secreted from the cells. The imaging container may centrifuged to align cells along the imaging surface or the cells may be allowed to settle on the imaging surface of the imaging container without centrifugation; 3) Cross-linking the pre-hydrogel polymer solution by chemical and/or photo activation to create a polymerized hydrogel; 4) Waiting an appropriate amount of time to allow the cells to secrete molecules; 5) Applying reagents, such as for fixation, permeabilization, and staining along with appropriate washing steps, to stain the cells and the captured secreted molecules within the polymerized hydrogel; 6) Imaging to determine the phenotype for each cell, as well as the identity and amount of cell-secreted molecules captured within the hydrogel.

Advantageously, the compositions and method described herein offer reduced cell loss compared to alternative approaches. The compositions and methods described herein may facilitate laboratory techniques such as ICC by providing an antibody-permeable hydrogel to constrain encapsulated cells to an imaging surface for ICC, thereby reducing the requirement for additional centrifugation steps.

Various embodiments and examples of the invention are described herein. These embodiments and examples are illustrative and should not be construed as limiting the scope of the invention.

### Materials and Methods

Chemicals and hydrogel preparation: The hydrogels PEG700DA, PEG6000DA, PEG10000DA, PEG 20000 (Mw 20000 Da), photo initiator ‘2-Hydroxy-4′-(2-hydroxyethoxy)-2-methylpropiophenone’ (or Irgacure™ 2959), paraformaldehyde (PFA), and Tween-20 were all purchased from Sigma-Aldrich™, Canada. Different formulations of PEGDAs diluted in phosphate buffered saline (PBS) were tested for their various properties, which included curing time, mechanical stability, and staining time. The hydrogel macromer solution selected for the lossless experiments was prepared at 30% (w/v) of PEG700DA in PBS and 30% (w/v) of PEG 20000 in PBS. Photo-initiator was mixed at 1% (w/v) in 100% ethanol. The solution was then diluted with the cell suspension, such that their final concentration was 15% (w/v) of PEG700DA, 15% (w/v) of PEG 20,000, and 0.1% (w/v) of photo-initiator to form the pre-hydrogel polymer solution. Each solution was freshly prepared prior to experiments.

Cell culture: The cell line 22RV1 (human prostate carcinoma) was used for validation experiments. Cells were maintained in RPMI-1640 culture media containing 10% Fetal Bovine Serum (Gibco™) and 1% penicillin-streptomycin (Gibco™) at 5% CO₂ at 37° C. Cells were re-suspended using 0.25% Trypsin-EDTA (Gibco™) and were serially diluted to 10,000, 1,000, 100 and 10 cells per 40 μl culture media.

Cell encapsulation: To encapsulate the cells in hydrogel, the cell suspensions and 40 μL of PBS buffer were loaded into wells of a 384-high contrast imaging well-plate (Corning™) with 6.5 μL of the premixed pre-hydrogel polymer solution. The imaging well-plate was centrifuged for 3 minutes at 3800 rpm, followed by exposure to 375 nm high-power UV LED (Thorlabs™) for 5 seconds.

Cytospin™: Cytospin™ was performed by spinning a 40 μL cell suspension directly onto a BSA-coated glass slide using a cytocentrifuge (Cytospin™ 2, Shandon) at 700 rpm for 3 minutes with low acceleration.

Immunocytochemistry: To validate ICC on the encapsulated cells, 3 common imaging reagents for cancer cell identification were used; DAPI (1 μM) for DNA, EpCam-Alexafluor-488 for surface staining of the epithelial cell adhesion molecule present on the cell membrane and Pan-Keratin-Alexafluor-647 (1:100 dilution) to intracellularly stain cytokeratin which is present in the cell cytoplasm. ICC was performed in parallel on matching samples of non-encapsulated cells in the imaging plate, encapsulated cells in the imaging plate and cells that were cytospun onto a glass slide. For intracellular staining cells were fixed in 4% PFA for 10 minutes, followed by two PBS washes and then permeabilized with 0.025% Tween-20 for 15 minutes followed by two washes. A 3% BSA solution was applied as a blocking agent for 30 minutes, after which the antibodies were added and incubated for 1 hour. For staining non-encapsulated cells in the imaging plate, washes were done by adding 40 μl of PBS followed by centrifugation at 3800 rpm for 3 minutes. Washing the Cytospin™ slides involved rinsing them in PBS, while washing hydrogel encapsulated cells involved adding PBS and pipetting up and down about 10 times per wash. After washing unbound antibodies, the cells were directly imaged using both bright field and fluorescent microscopy, using a Nikon™ Ti-E inverted fluorescent microscope with 10×, 20× and 60× magnification with a high-resolution camera or a Zeiss™ laser scanning confocal microscope LSM 780 at 40× magnification.

Cell counting and statistical analysis: Both the initial (prior to plating) and final numbers of all 3 matching ICC samples were manually counted by two individuals from the obtained images using ImageJ™ software. Experiments were performed 3 times for each cell dilution. Results from the count were averaged and plotted using Graphpad™ Prism software.

### EXAMPLES

The following examples are provided for illustrative purposes, and are not intended to be limiting, as such.

### Example 1. Optimization of Hydrogel Cell Encapsulation Compositions

To prevent damage to the cells and their DNA, a photo-initiator, Irgacure™ 2959, was selected based on its transparency and ability to absorb long wave UV light (>350 nm). To reduce cytotoxicity, the concentration of Irgacure™ 2959 was limited to 0.1% (w/v). However, an alternative cross-linking agent may be used provided and depending on the crosslinking agent chosen may be used at a greater concentration. The thickness, porosity, and mechanical stability of the PEGDA hydrogel can be optimized either by varying their molecular weight or by mixing with poly (ethylene glycol) (PEG) and PBS. The hydrogel porosity can be optimized to encapsulate and affix cells to the surface of an imaging well plate, while allowing antibodies to diffuse through the pores and reach the cells. The mechanical stability of the photo-polymerized hydrogel is important to withstand pipette manipulation during the staining process while the thickness of the hydrogel should allow for reagents to reach the encapsulated cells via diffusion. The effects of these parameters on the properties of PEGDA hydrogels are summarized in TABLE 3A.

TABLE 3A
PROPERTIES OF TESTED PEGDA HYDROGELS

| | | | | Staining |
|---|---|---|---|---|
| | Proportion | | Mechanical | Time |
| Type of PEGDA | (% w/v) | Curing Time (s) | Stability* | (hrs) |
| PEGDA average | water insoluble | | | |
| Mₙ 250 | | | | |
| PEGDA average | 100 | <1 | >100 | n/a |
| Mₙ 575 | 80 | <1 | >100 | n/a |
| | 50 | 2 | >100 | n/a |
| | 30 | 3 | >100 | n/a |
| | 15 | 5 | >100 | n/a |
| PEGDA average | 15/30 | U* | | |
| Mₙ 575/PEG | 15/15 | 5 | >100 | n/a |
| average Mₙ | 15/5 | 5 | >100 | n/a |
| 20,000 | | | | |
| PEGDA average | 100 | <1 | >100 | 24 |
| Mₙ 700 | 50 | 2 | >100 | 12 |
| | 30 | 3 | >100 | 8 |
| | 15 | 5 | >100 | 4 |
| | 5 | U* | — | — |
| PEGDA average | 15/30 | U* | — | — |
| Mₙ 700/PEG | 15/15 | 5 | >100 | 1 |
| average Mₙ | 15/5 | 7 | >100 | 4 |
| 20,000 | | | | |
| PEGDA average | Not tested | | | |
| Mₙ 1,000 | | | | |
| PEGDA average | Not tested | | | |
| Mₙ 2,000 | | | | |
| PEGDA average | 80 | 2 | 10 | 12 |
| Mₙ 6,000 | 50 | 5 | 1 | — |
| | 30 | Uncured | — | — |
| | 15 | Uncured | — | — |
| | 5 | Uncured | — | — |
| PEGDA average | 80 | 3 | <5 | 12 |
| Mₙ 10,000 | 50 | 5 | 1 | — |
| | 30 | Uncured | — | — |
| | 15 | Uncured | — | — |
| | 5 | Uncured | — | — |

U*: these polymer solutions did not cure using 0.1% w/v Irgacure ™ 2959, 375 nm UV with up to a 5 min exposure. However, using 1% w/v Irgacure ™ 2959 and 365 nm UV, it was possible to cure the polymer solutions in <1 min.
*mechanical stability was measured as the number of pipettings of 40 μls of PBS at of 80 μl/s through a 200 μl pipette tip (with an opening bore of 460 μm) without significant structural disintegration (i.e. cracks, tears, delamination of the thin layer hydrogel formed after crosslinking). A lower limit of mechanical stability of about 10 was considered necessary to withstand ICC addition and washings.
Staining time above measured by imaging the cells in given time frame (1, 2, 4, 8, 12, 24 hours). Once most cells (around 95%) shows similar brightness that doesn't encapsulated stained cells (i.e. cells stained by common ICC protocol) considered as stained.

TABLE 3B
PROPERTIES OF TESTED MODIFIED PEG HYDROGELS WITH DENSITY (g/ml)

| | Concentration | Density | Curing | Mechanical |
|---|---|---|---|---|
| Type(s) of PEG derivative | (% w/v in PBS) | (g/ml) | Time (s) | Stability* |
| PEGDA average Mₙ 575 | 100 | 1.12 | <1 | >100 |
| | 80 | 1.096 | <1 | >100 |
| | 50 | 1.06 | 2 | >100 |
| | 30 | 1.036 | 3 | >100 |
| | 15 | 1.018 | 5 | >100 |
| PEGDA average Mₙ 575/PEG | 15/30 | 1.099 | U* | |
| average Mₙ 20,000 | 15/15 | 1.058 | 5 | >100 |
| | 15/5 | 1.032 | 5 | >100 |
| PEGDMA average Mₙ 550 | 100 | 1.1 | 3 | <30 |
| | 80 | 1.08 | 3 | <30 |
| | 50 | 1.05 | 5 | <20 |
| | 30 | 1.03 | 5 | <20 |
| | 15 | 1.015 | 5 | <20 |
| | 5 | 1.005 | 10 | <10 |
| PEGDMA average Mₙ 550/PEG | 15/30 | 1.096 | 15 | <5 |
| average Mₙ 20,000 | 15/15 | 1.056 | 10 | <10 |
| | 15/5 | 1.029 | 10 | <20 |
| PEGMA average Mₙ 360 | 100 | 1.08 | 25 | >100 |
| | 80 | 1.064 | 25 | >100 |
| | 50 | 1.04 | 25 | >100 |
| | 30 | 1.024 | 50 | <20 |
| | 15 | 1.012 | 50 | <20 |
| | 5 | 1.004 | U* | |
| PEGMA average Mₙ 360/PEG | 15/30 | 1.093 | U* | |
| average Mₙ 20,000 | 15/15 | 1.053 | 90 | <20 |
| | 15/5 | 1.026 | 90 | <20 |
| HEMA average Mₙ 130 | 100 | 1.08 | U* | |
| | 80 | 1.064 | U* | |
| | 50 | 1.04 | U | |
| | 30 | 1.024 | U | |
| | 15 | 1.012 | U | |
| | 5 | 1.004 | U | |
| PEGMEA average Mₙ 500 | 100 | 1.05 | U* | |
| | 80 | 1.04 | U | |
| PEGMEA average Mₙ 300 | 100 | 1.05 | U* | |
| | 80 | 1.04 | U | |

U*: see above for TABLE 3A.
*mechanical stability-see above for TABLE 3A.
∞Some commercially available modified PEG polymers have variability in the degree to which termini are modified and this may account for variability in the ability of the scaffold polymers to cross-link to one another and could result in reduced mechanical stability or even inability to cure into a hydrogel. Alternatively, additional co-polymers could be used to facilitate cross-linking and hydrogel formation.

The ratios of scaffold polymer:porogen may be estimated for any combination of scaffold polymer to porogen depending on the particular cell type to be encapsulated. For example, the below TABLES 4A-4D show ratios optimized for monocytes (i.e. between about 1.067 g/ml about 1.077 g/ml). Please see the attached for the estimated polymer density for different mixtures of PEGDA 700, 575, 500, 360, and Gel-MA 45k all mixed with PEG 20k. In most cases the maximum density was set at 1.067, but any other maximum density could be achieved depending on the cells to be encapsulated.

TABLE 4A
Estimated Polymer Density for Different Mixtures of PEGDA
Average Mₙ 575/PEG Average Mₙ 20,000

| Type(s) of PEG derivative | Concentration (% w/v in PBS) | Density (g/ml) |
|---|---|---|
| PEGDA (Mw575)/PEG | 44.5/5 | 1.066 |
| (Mw20k) | 40/5 | 1.062 |
| or PEGDA (Mw 700)/PEG | 35/5 | 1.056 |
| (Mw20k) | 30/5 | 1.05 |
| | 25/5 | 1.044 |
| | 20/5 | 1.038 |
| | 15/5 | 1.032 |
| | 10/5 | 1.026 |
| | 5/5 | 1.020 |
| | 33/10 | 1.067 |
| | 30/10 | 1.063 |
| | 25/10 | 1.057 |
| | 20/10 | 1.051 |
| | 15/10 | 1.045 |
| | 10/10 | 1.039 |
| | 5/10 | 1.033 |
| | 22/15 | 1.067 |
| | 20/15 | 1.065 |
| | 15/15 | 1.059 |
| | 10/15 | 1.053 |
| | 5/15 | 1.047 |
| | 11/20 | 1.067 |
| | 10/20 | 1.066 |
| | 5/20 | 1.06 |

Note:
shaded indicate maximum possible density. Since lowest density cells like monocytes are between 1.067~1.077 g/ml.

TABLE 4B
Estimated Polymer Density for Different Mixtures of PEGDMA Average
Mₙ 550/PEG Average Mₙ 20,000

| Type(s) of PEG derivative | Concentration (% w/v in PBS) | Density (g/ml) |
|---|---|---|
| PEGDMA (Mw550)/PEG | 53/5 | 1.067 |
| (Mw20k) | 50/5 | 1.064 |
| | 45/5 | 1.059 |
| | 40/5 | 1.054 |
| | 35/5 | 1.049 |
| | 30/5 | 1.044 |
| | 25/5 | 1.039 |
| | 20/5 | 1.034 |
| | 15/5 | 1.029 |
| | 10/5 | 1.024 |
| | 5/5 | 1.019 |
| | 40/10 | 1.067 |
| | 35/10 | 1.062 |
| | 30/10 | 1.057 |
| | 25/10 | 1.052 |
| | 20/10 | 1.047 |
| | 15/10 | 1.042 |
| | 10/10 | 1.037 |
| | 5/10 | 1.032 |
| | 26/15 | 1.067 |
| | 25/15 | 1.066 |
| | 20/15 | 1.061 |
| | 15/15 | 1.056 |
| | 10/15 | 1.051 |
| | 5/15 | 1.046 |
| | 13/20 | 1.067 |
| | 10/20 | 1.064 |
| | 5/20 | 1.059 |
| | 10/21 | 1.067 |

TABLE 4C
Estimated Polymer Density for Different Mixtures of PEGMA Average
Mₙ 360/PEG Average Mₙ 20,000

| Type(s) of PEG derivative | Concentration (% w/v in PBS) | Density (g/ml) |
|---|---|---|
| PEGMA (Mw360)/PEG | 65/5 | 1.066 |
| (Mw20k) | 60/5 | 1.062 |
| | 55/5 | 1.058 |
| | 50/5 | 1.054 |
| | 45/5 | 1.05 |
| | 40/5 | 1.046 |
| | 35/5 | 1.042 |
| | 30/5 | 1.038 |
| | 25/5 | 1.034 |
| | 20/5 | 1.03 |
| | 15/5 | 1.026 |
| | 10/5 | 1.022 |
| | 5/5 | 1.018 |
| | 50/10 | 1.067 |
| | 45/10 | 1.063 |
| | 40/10 | 1.059 |
| | 35/10 | 1.055 |
| | 30/10 | 1.051 |
| | 25/10 | 1.047 |
| | 20/10 | 1.043 |
| | 15/10 | 1.039 |
| | 10/10 | 1.035 |
| | 5/10 | 1.031 |
| | 30/15 | 1.065 |
| | 25/15 | 1.061 |
| | 20/15 | 1.057 |
| | 15/15 | 1.053 |
| | 10/15 | 1.049 |
| | 5/15 | 1.045 |
| | 15/20 | 1.066 |
| | 10/20 | 1.062 |
| | 5/20 | 1.058 |
| | 10/21 | 1.065 |

TABLE 4D
Estimated Polymer Density for Different Mixtures of Gelatin-MA Average
Mₙ 360/PEG Average Mₙ 20,000

| Type(s) of PEG derivative | Concentration (% w/v in PBS) | Density (g/ml) |
|---|---|---|
| Gelatin-MA (Mw 45k)/ | 20/5 | 1.054 |
| PEG (Mw 20k) | 15/5 | 1.044 |
| | 10/5 | 1.034 |
| | 5/5 | 1.024 |
| | 1/5 | 1.016 |
| | 20/10 | 1.067 |
| | 15/10 | 1.057 |
| | 10/10 | 1.047 |
| | 5/10 | 1.037 |
| | 1/10 | 1.029 |
| | 10/15 | 1.061 |
| | 5/15 | 1.051 |
| | 1/15 | 1.043 |
| | 5/20 | 1.064 |
| | 1/20 | 1.056 |

Hydrogel Porosity: In order to optimize the hydrogel for cell encapsulation, it is important to control the PEGDA hydrogel porosity since it controls several key properties relevant to ICC, including swelling (thickness), antibody diffusivity, and mechanical stability¹⁵. Macro-porous hydrogels (˜>100 μm) are often used for tissue engineering applications, such as providing three-dimensional cell culture platforms for tissue regeneration^{16,17}. The large pore sizes allows sufficient space for cell growth and vascularization, as well as the capacity to retain required cell nutrients while allowing the diffusion of metabolic waste¹⁸⁻²⁰. However, the methods used to create macro-porous hydrogels such as freeze-drying, solvent casting, and gas formation that combine with cross-linking of the hydrogel²¹⁻²⁶, can cause severe damage to the cell. Consequently, cells are typically seeded on the surface of pre-formed gels, and then allowed to grow into the internal cavities of the gel. Although cells are inside the hydrogels, they are not encapsulated because there are only minimal points of contact between the cell membrane and the hydrogel, allowing cell movement Therefore, micro-porous hydrogels (up to 10 nm) are preferred for therapeutic applications, because they can provide similar features to macro-porous hydrogels, but they can also protect encapsulated cells from the infiltrating immune system, such as in the case of encapsulation of genetically modified cytokine-secreting cells that are implanted into tumors to coordinate the anti-tumor immune response²⁷. However, for the current application, micro-porous hydrogels would prevent reagents such as large proteins (IgG, etc.) from diffusing through and reaching encapsulated cells. Hence, a hydrogel porosity that encapsulates cells while allowing reagents to diffuse through the pores and reach the cells is the goal of the present compositions.

In order to enable diffusion of large proteins through hydrogel, different formulations of PEGDA and other scaffold polymers were investigated. Hydrogels with different pore sizes were generated by varying their molecular mass by dilution in PBS (TABLE 3A). However, while it is easy to alter the pore sizes of PEGDA hydrogels by either changing the molecular weights of PEG chains in the macromer or by altering the macromer concentration in solution, the pore size is still limited to approximately 50 nm under thin film^{28,29}. In this range, large proteins such as IgG (150 kDa, ˜70 nm) cannot diffuse through²⁸ and it is thus ineffective for ICC. Several studies have reported small-molecule diffusion in hydrogels made from concentrated solutions (>50%) of PEGDA³⁰⁻³⁴ and diffusion of proteins has also been studied in PEG hydrogels with >10% polymer content^{28, 35-37}. Consequently, the effects of PEG as a porogen on PEGDA hydrogel structures has been investigated to improve macromolecular diffusion in biological applications that require transport of large solutes through hydrogels²⁹.

PEG porogens function to increase the heterogeneity of polymerization areas. During photo-polymerization, the activation of the photo-initiator releases free-radicals which attack the acrylate end of PEGDA, and rapidly form multiple localized polymer chain clusters. These chain clusters continue to grow as long as the free-radicals exist, thus forming a complete polymer. The polymerization of diacrylates forms heterogeneous gels that have areas of high cross-link densities surrounded by areas of low cross-link densities^{38,39}. The PEG porogens increase the density heterogeneity of the diacrylate monomers by pooling in areas that are then excluded from crosslinking. An added washing step would remove these areas resulting in a lower overall cross-linking density and a higher porosity hydrogel²⁹. Furthermore, by adjusting the light intensity, the polymer chain clusters can be controlled. At low light intensity, phase-separation of the PEG and PEGDA can occur, allowing for large polymer clusters to grow, which increases the pore size. Therefore, by increasing the light intensity, targeted pore sizes can be achieved with the use of appropriate molecular weights of PEG.

High molecular weight PEG (PEG average Mn 20,000) was therefore employed as a porogen for PEG700DA (PEGDA average Mn 700) to increase the precision of the pore size to better allow diffusion of antibodies for ICC. A 1:1 mixture of PEG700DA to PEG 20000, each at 15% (w/v), with a 0.1% (w/v) final concentration of photo-initiator, in PBS, was used to generate an ICC stable hydrogel that allowed cells to be encapsulated and staining reagents to reach the cells in a relatively short time (as measured by the staining time in TABLES 3A and 3B).

Hydrogel mechanical stability: The mechanical strength of the hydrogel thin-film is important for retaining structural integrity during pipetting. This property was tested by repeatedly pipetting 40 μl of PBS onto the surface of the photopolymerized hydrogel multiple times until signs of structure disintegration, such as cracks, tears, delamination of the hydrogel thin-film, were observed. As shown in TABLES 3A and 3B, PEG6000-DA and PEG10000-DA formulations were structurally weaker and could only survive a few rounds of pipetting even at low dilution. On the other hand, PEG700-DA, even at low dilution, had sufficient mechanical strength to survive pipetting 40 μl of PEGDA more than 100 times.

Hydrogel Thickness: The thickness of the hydrogel thin-film can affect the amount of time required for reagents, including antibodies, to diffuse through the film and reach the encapsulated cells. The thickness of the hydrogel thin-film can be controlled by the intensity of UV light, exposure time, and the concentration and spectral characteristics of the photo-initiator used to polymerize the hydrogel. Light penetration through the PEGDA hydrogel can be estimated using the Beer-Lambert law,

$$T = \frac{{Φ}_{e}^{t}}{{Φ}_{e}^{i}} = {e}^{- τ} = 1 {0}^{- A} ,$$

where the transmittance (T) of material sample is related to its optical depth (τ) and to its absorbance (A), as Φₑ^{t} is the radiant flux transmitted by that material sample; and Φₑⁱ is the radiant flux received by that material sample. This equation shows that the light intensity is exponentially decreasing as it penetrates the material due to absorption. Ideally, it is possible to calculate the light intensity at a certain depth. However, this equation can only explain the decreasing light intensity, and not the actual polymerizing depth due to the presence of free-radicals which propagates the polymerization, therefore, the final thickness is not only intensity-dependent but also time-dependent.

The thickness of a 1:1 mixture of PEG700DA to PEG 20000, each at 15% (w/v) using 5 seconds' exposure time to 375 nm UV light, was measured to be ˜100 μm. Thickness was measured using a microscope and changing the focal distance from the bottom of the imaging plate, which focused on the cell, to the top of the hydrogel layer, using a 60× objective.

### Example 2. Staining and Image Acquisition Using ICC Composition

To investigate the efficiency of ICC stain as well as image quality of encapsulated cells, we used a standard ICC protocol, according to the manufacturer's guideline⁴⁰, for staining cells and compared the staining of encapsulated cells to non-encapsulated cells. However, instead of using centrifugation to remove the excess antibody stains, supernatant from each washing step may simply be removed by pipetting. Image acquisition in macroporous hydrogels, after polymerization, has traditionally proven to be difficult due to the large pore sizes²⁹. To determine if the PEG porogen influences image quality, we imaged encapsulated cells before and after photo-polymerization. Prior to polymerization, the hydrogel was transparent, but became lightly opaque after photo-polymerization. However, this color change had no effect on the visualization of unstained or stained cells by bright field microscopy (data not shown). The comparison of PEGDA hydrogels before and after photo-polymerization compared macroscopic images of a single 384 well with PEGDA before photo-polymerization with a macroscopic image of a single 384 well after PEGDA hydrogel is photo-polymerized. Bright field microscopic images of single well plate before photo-polymerization and bright field microscopic images of the same well, were compared before and after photo-polymerization, hydrogel become lightly opaque but there was no significant change in image quality for microscopy noted.

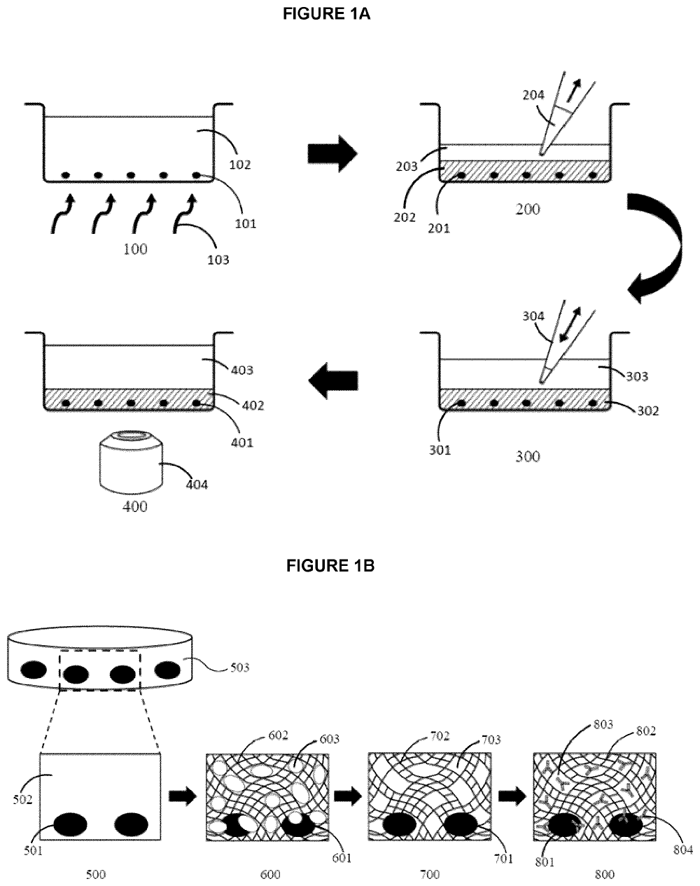

Encapsulated stained cells (see FIGS. 1A and 1B) can be directly imaged using multi-colour fluorescent images without compromising the staining efficiency (images not shown). Using this method, staining can be done in a comparable amount of time to standard ICC (<2 hours). Furthermore, there is no background fluorescence, which indicates that unbound antibodies were washed away and that non-specific binding between antibody and the hydrogel network was minimal. Once hydrogel-encapsulated, the cells were then stained with fluorescent markers. In one example, the scanned well plate image from encapsulating 1000 cells with 3 fluorescent channels merged or visualized individually and when magnified individual cells could easily be visualized with the 3 separate fluorescent channels tested (i.e. Blue—DAPI, Green—EpCam-Alexafluor-488, and Red—Pan-Keratin-Alexafluor-647).

### Example 3: Quantification of Cell Loss in Immunocytochemistry

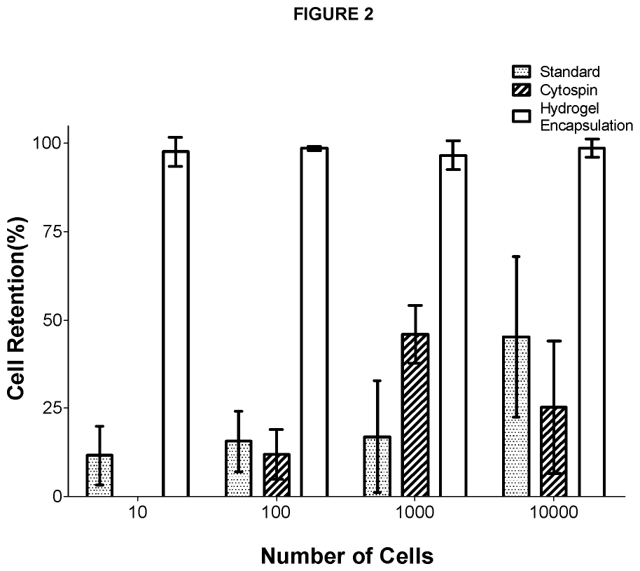

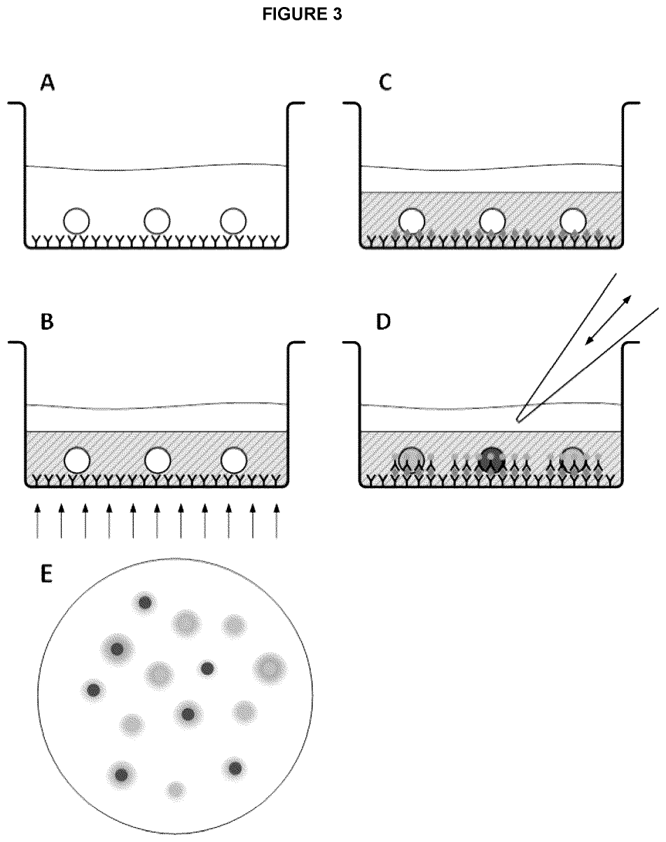

To quantify cell loss during ICC, cells were counted before and after ICC for sample sizes of 10, 100, 1,000, and 10,000 cells using three different protocols: 1) traditional ICC performed on 384-well imaging plates, 2) ICC performed on cells adhered to microscope slides using cytospin, and 3) ICC performed on PEGDA hydrogel encapsulated cells. Two individuals counted encapsulated cells in each image and the results were averaged to limit any error resulting from manual counting. Traditional ICC and CytoSpin™ showed a staggering amount of cell loss for cell samples ranging from 10 cells to 10,000 cells (FIG. 2). On the other hand, the current cell encapsulating hydrogel ICC compositions and methods limited cell loss to 1-3% showing improved cell retention during staining, washing, and centrifugation for all sample sizes.

Although embodiments described herein have been described in some detail by way of illustration and example for the purposes of clarity of understanding, it will be readily apparent to those of skill in the art in light of the teachings described herein that changes and modifications may be made thereto without departing from the spirit or scope of the appended claims. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as herein described and with reference to the figures.

### REFERENCES

(1) Moumouni, H.; Garaud, P.; Diot, P.; Lemarie, E.; Anthonioz, P. Am. J. Respir. Crit. Care Med. 1994, 149 (3 Pt 1), 636-640.
(2) Gabor, F.; Hoffmann, O.; Pittner, F.; Wirth, M. In Analytical Biotechnology; Schalkhammer, T. G. M., Ed.; Birkhäauser Basel, 2002; p 311.
(3) de Bono, J. S.; Scher, H. I.; Montgomery, R. B.; Parker, C.; Miller, M. C.; Tissing, H.; Doyle, G. V; Terstappen, L. W. W. M.; Pienta, K. J.; Raghavan, D. Clin. Cancer Res. 2008, 14 (19), 6302-6309.
(4) Cristofanilli, M.; Budd, G. T.; Ellis, M. J.; Stopeck, A.; Matera, J.; Miller, M. C.; Reuben, J. M.; Doyle, G. V; Allard, W. J.; Terstappen, L. W. M. M.; Hayes, D. F. N. Engl. J. Med. 2004, 351 (8), 781-791.
(5) Cohen, S. J.; Punt, C. J. A.; Jannotti, N.; Saidman, B. H.; Sabbath, K. D.; Gabrail, N. Y.; Picus, J.; Morse, M.; Mitchell, E.; Miller, M. C.; Doyle, G. V; Tissing, H.; Terstappen, L. W. M. M.; Meropol, N. J. J. Clin. Oncol. 2008, 26 (19), 3213-3221.
(6) Miller, M. C.; Doyle, G. V; Terstappen, L. W. M. M. J. Oncol. 2010, 2010, 617421.
(7) Hamada, H.; Arinami, T.; Kubo, T.; Hamaguchi, H.; Iwasaki, H. Hum. Genet. 1993, 91 (5), 427-432.
(8) Masuda, H.-T.; Ishihara, S.; Harada, I.; Mizutani, T.; Ishikawa, M.; Kawabata, K.; Haga, H. Biotechniques 2014, 172 (56), 172-179.
(9) Srinivasan, S. Protoc. Exch. 2011.
(10) Allen, L.-A. H. Methods Mol. Biol. 2014, 1124, 251-268.
(11) Koh, C. M. In Methods in enzymology; 2013; Vol. 533, pp 235-240.
(12) Krishnamurthy, V.; Satish, S.; Doreswamy, S. M.; Vimalambike, M. G. J. Clin. Diagn. Res. 2016, 10 (7), EC18-20.
(13) Dhar, M.; Pao, E.; Renier, C.; Go, D. E.; Che, J.; Montoya, R.; Conrad, R.; Matsumoto, M.; Heirich, K.; Triboulet, M.; Rao, J.; Jeffrey, S. S.; Garon, E. B.; Goldman, J.; Rao, N. P.; Kulkarni, R.; Sollier-Christen, E.; Di Carlo, D. Nat. Publ. Gr. 2016.
(14) Nakayama, T.; Mihara, K.; Kawata, J.; Kimura, H.; Saitoh, H. Anal. Biochem. 2014, 466, 1-3.
(15) Peppas, N. A.; Hilt, J. Z.; Khademhosseini, A.; Langer, R. Adv. Mater. 2006, 18 (11), 1345-1360.
(16) Annabi, N.; Nichol, J. W.; Zhong, X.; Ji, C.; Koshy, S.; Khademhosseini, A.; Dehghani, F. Tissue Eng. Part B 2010, 16 (4), 371-383.
(17) Raic, A.; Rodling, L.; Kalbacher, H.; Lee-Thedieck, C. Biomaterials. 2014, pp 929-940.
(18) Roy, T. D.; Simon, J. L.; Ricci, J. L.; Rekow, E. D.; Thompson, V. P.; Parsons, J. R. J. Biomed. Mater. Res. 2003, 66A (2), 283-291.
(19) Kim, H. J.; Kim, U.-J.; Vunjak-Novakovic, G.; Min, B.-H.; Kaplan, D. L. Biomaterials 2005, 26 (20), 4442-4452.
(20) Griffon, D. J.; Sedighi, M. R.; Schaeffer, D. V.; Eurell, J. A.; Johnson, A. L. Acta Biomater. 2006, 2 (3), 313-320.
(21) Beamish, J. A.; Zhu, J.; Kottke-Marchant, K.; Marchant, R. E. J. Biomed. Mater. Res. A 2010, 92 (2), 441-450.
(22) Van Den Bulcke, a I.; Bogdanov, B.; De Rooze, N.; Schacht, E. H.; Cornelissen, M.; Berghmans, H. Biomacromolecules 2000, 1 (1), 31-38.
(23) Artyukhov, A. A.; Shtilman, M. I.; Kuskov, A. N.; Pashkova, L. I.; Tsatsakis, A. M.; Rizos, A. K. In Journal of Non-Crystalline Solids; 2011; Vol. 357, pp 700-706.
(24) Gemeinhart, R. a.; Park, H.; Park, K. Polym. Adv. Technol. 2000, 11 (8-12), 617-625.
(25) Hoffman, A. S. Adv. Drug Deliv. Rev. 2012, 64, 18-23.
(26) Ahmed, E. M. J. Adv. Res. 2015, 6 (2), 105-121.
(27) Cirone, P.; Bourgeois, J. M.; Austin, R. C.; Chang, P. L. Hum. Gene Ther. 2002, 13 (10), 1157-1166.
(28) Cruise, G. M.; Scharp, D. S.; Hubbell, J. A. Biomaterials 1998, 19 (14), 1287-1294.
(29) Lee, A. G.; Arena, C. P.; Beebe, D. J.; Palecek, S. P. Biomacromolecules 2010, 11 (12), 3316-3324.
(30) Watkins, A. W.; Anseth, K. S. Macromolecules 2005, 38 (4), 1326-1334.
(31) Tomić, K.; Veeman, W. S.; Boerakker, M.; Litvinov, V. M.; Dias, A. A. J. Pharm. Sci. 2008, 97 (8), 3245-3256.
(32) Scott, R. A.; Peppas, N. A. Biomaterials 1999, 20 (15), 1371-1380.
(33) Rounds, R. M.; Ibey, B. L.; Beier, H. T.; Pishko, M. V.; Coté, G. L. J. Fluoresc. 2007, 17 (1), 57-63.
(34) Iza, M.; Stoianovici, G.; Viora, L.; Grossiord, J. L.; Couarraze, G. J. Control. Release 1998, 52 (1-2), 41-51.
(35) Weber, L. M.; Lopez, C. G.; Anseth, K. S. J. Biomed. Mater. Res. Part A 2009, 90A (3), 720-729.
(36) Van De Wetering, P.; Metters, A. T.; Schoenmakers, R. G.; Hubbell, J. A. J. Control. Release 2005, 102 (3), 619-627.
(37) Sperinde, J. J.; Griffith, L. G. Macromolecules 1997, 30 (18), 5255-5264.
(38) Kannurpatti, A. R.; Bowman, C. N. Macromolecules 1998, 31 (10), 3311-3316.
(39) Simon, G. P.; Allen, P. E. M.; Williams, D. R. G. Polymer (Guildf). 1991, 32 (14), 2577-2587.
(40) A guide to successful immunofluorescence http://www.neb-online.de/wp-content/uploads/2017/03/CST_A_Guide_to_Successful_Immunofluorescence_web.pdf.
(41) R. Long and C. Hui, “Fracture toughness of hydrogels: measurement and interpretation”, Soft Matter, vol. 12, no. 39, pp. 8069-8086, 2016.
(42) J. Tang J. Li, J. Vlassakand Z. Suo, “Fatigue fracture of hydrogels”, Extreme Mechanics Letters, vol. 10, pp. 24-31, 2017.
(43) Zipursky, A.; Bow, E.; Seshardi, R. S. Brown, E. J. Blood 1976, 48 (3), 361-371.
(44) Grover, W. H. et al. PNAS 2011 108(27):10992-10996.
(45) Pellegrini, M. et al. Tissue Engineering 2015 21(12):1246-1250.
(46) Kovac, J. et al. Adv. Healthcare Mater. 2013 2:552-556.

1. A composition, the composition comprising:
(a) a scaffold polymer, wherein the scaffold polymer:
(i) has one or more acryloyl group or one or more methacryloyl groups;
(ii) has an average molecular weight (Mₙ) between about 300 and about 6,000;
(iii) is water soluble and biocompatible; and
(iv) is operable to form a hydrogel following cross-linking;
(b) a porogen; and
(c) a crosslinking agent;
wherein, the composition has a density of between about 1.0 g/ml and about 1.12 g/ml at 25° C.

2. The composition of claim 1, wherein composition has a density of between about 1.0 g/ml and about 1.10 g/ml at 25° C.

3. The composition of claim 1 or 2, wherein composition has a density of between about 1.0 g/ml and about 1.08 g/ml at 25° C.

4. The composition of claim 1, 2 or 3, wherein scaffold polymer has an average molecular weight (Mₙ) between about 300 and about 3,000.

5. The composition of any one of claims 1-4, wherein the scaffold polymer is selected from the following: Poly(ethylene glycol) diacrylate (PEGDA); Poly(ethylene glycol) dimethylacrylate (PEGDMA); Poly(ethylene glycol) methyl ether acrylate (PEGMEA); Poly(ethylene glycol) methacrylate (PEGMA); and Poly(ethylene glycol) methyl ether methacrylate (PEGMEMA).

6. The composition of any one of claims 1-5, wherein the scaffold polymer is selected from the following: PEGDA; PEGDMA; PEGMA; and PEGMEMA.

7. The composition of any one of claims 1-6, wherein the scaffold polymer is selected from the following: PEGDA and PEGDMA.

8. The composition of any one of claims 1-7, wherein the scaffold polymer is PEGDA.

9. The composition of any one of claims 1-8, wherein the scaffold polymer has an average Mₙ between about 300 and about 6,000.

10. The composition of any one of claims 1-9, wherein the scaffold polymer has an average Mₙ between about 300 and about 2,000.

11. The composition of any one of claims 1-10, wherein the scaffold polymer has an average Mₙ of about 700.

12. The composition of any one of claims 2-11, wherein the porogen is selected from one or more of the following: Poly(ethylene glycol) (PEG); Chitosan; Agarose; Dextran; Hyaluronic acid; Poly(methyl methacrylate) (PMMA); Cellulose and derivatives thereof; Gelatin and derivatives thereof; and Acrylamide and derivatives thereof.

13. The composition of any one of claims 2-12, wherein the porogen is PEG.

14. The composition of any one of claims 2-13, wherein the porogen is PEG and has an average Mₙ between 1,000 and 40,000.

15. The composition of any one of claims 2-14, wherein the porogen is PEG and has an average Mₙ of 20,000.

16. The composition of any one of claims 2-15, wherein:
(i) the weight ratio of the scaffold polymer to porogen is about 1:1;
(ii) the scaffold polymer is PEGDA having an average Mₙ of 700 and 15% w/v; and
(iii) the porogen is PEG having an average Mₙ of 20,000 and 15% w/v.

17. The composition of any one of claims 1-16, wherein the crosslinking agent is a free-radical generating compound.

18. The composition of any one of claims 1-16, wherein the crosslinking agent is a photo-initiator [UV] selected from TABLE 1B.

19. The composition of any one of claims 1-18, wherein the crosslinking agent is Irgacure 819 or Irgacure 2959.

20. The composition of any one of claims 1-19, wherein the crosslinking agent is Irgacure 2959 at 0.1% w/v or Irgacure 819 at 0.1% w/v.

21. A composition, the composition comprising:
(a) a scaffold polymer, wherein the scaffold polymer:
(i) is selected from: PEGDA; PEGMA; and PEGDMA;
(ii) has an average molecular weight (Mₙ) between about 500 and about 3,000;
(iii) is water soluble and biocompatible; and
(iv) is operable to form a hydrogel following cross-linking; and
(b) 2-Hydroxy-1-[4-(2-hydroxyethoxy)phenyl]-2-methyl-1-propanone is less than or equal to 1.0% w/v of the composition;
wherein, the composition has a density of between about 1.0 g/ml and about 1.10 g/ml at 25° C.

22. The composition of claim 21, wherein the composition further comprises a porogen.

23. The composition of claim 21 or 22, wherein the 2-Hydroxy-1-[4-(2-hydroxyethoxy)phenyl]-2-methyl-1-propanone is less than or equal to 0.3% w/v of the composition

24. The composition of claim 21, 22 or 23, wherein the 2-Hydroxy-1-[4-(2-hydroxyethoxy)phenyl]-2-methyl-1-propanone is less than or equal to 0.1% w/v of the composition

25. A cell encapsulation method, the method comprising:
(a) mixing a composition of claim 1-20 with a cells or a cell suspension to form a cell polymer mixture;
(b) adding the cell polymer mixture to a cell imaging container;
(c) settling the cell within the cell imaging container; and
(d) cross-linking the cell polymer mixture to form a hydrogel.

26. The method of claim 25, wherein the method further comprises assaying of the cells encapsulated by the hydrogel using immunocytochemistry.

27. The method of claim 25 or 26, wherein settling of the cell within the cell imaging container is by centrifugation.

28. The method of claim 26 or 27, the method further comprising bleaching the fluorescence from a previous immunocytochemistry assay and assaying of the cells encapsulated by the hydrogel using a second immunocytochemistry assay.

29. The method of claim 28, the method further comprising repeated bleaching of fluorescence and assaying of the cells encapsulated by the hydrogel using immunocytochemistry.

30. A cell encapsulation method, the method comprising:
(a) adding a crosslinking agent to the surface of a cell imaging container;
(b) adding a composition to the cell imaging container, the composition comprising:
(i) a scaffold polymer, wherein the scaffold polymer:
has one or more acryloyl group or one or more methacryloyl groups;
has an average molecular weight (Mₙ) between about 300 and about 6,000;
is water soluble and biocompatible; and
is operable to form a hydrogel following cross-linking; and
(ii) a porogen;
(c) adding cells or a cell suspension to the composition to form a cell polymer mixture in the imaging container;
(d) settling the cell within the cell imaging container; and
(e) cross-linking the cell polymer mixture to form a hydrogel.

31. The method of claim 30, wherein the method further comprises assaying of the cells encapsulated by the hydrogel using immunocytochemistry.

32. The method of claim 30 or 31, wherein the wherein settling of the cell within the cell imaging container is by centrifugation.

33. The method of claim 30 or 32, the method further comprising bleaching the fluorescence and assaying of the cells encapsulated by the hydrogel using immunocytochemistry.

34. The method of claim 33, the method further comprising bleaching the fluorescence from a previous immunocytochemistry assay and assaying of the cells encapsulated by the hydrogel using a second immunocytochemistry assay.

35. The method of any one of claims 30-34, wherein the hydrogel has a thickness of between about 10 μm and about 1,000 μm.

36. The method of any one of claims 30-35, wherein the hydrogel has pores between about 10 nm and about 10 μm.

37. The method of any one of claims 30-36, wherein the cross-linking is by UV light.

38. The method of any one of claims 30-37, wherein the cross-linking is by UV light at a wavelength between about 300 nm and about 375 nm.

39. The method of any one of claims 30-38, wherein the cross-linking is by UV light at a wavelength between about 300 nm and about 375 nm for an exposure of 5 seconds or less.

40. A cell encapsulation kit, the kit comprising:
(a) composition of any one of claims 1-24; and
(b) instructions for the compositions use in the encapsulation of cells.

41. The kit of claim 40, further comprising immunocytochemistry reagents.

42. The kit of claim 40 or 41, further comprising an imaging container.
